(19) United States
(12) Patent Application Publication
(10) Pub. No.: US 2003/0158503 A1
(43) Pub. Date: Aug. 21, 2003

(54) Capsule endoscope and observation system that uses it

(76) Inventor: Shinya Matsumoto, Tokyo (JP)

Correspondence Address: Arnold International, P.O. BOX 129, Great Falls, VA, 22066, US

(21) Appl. No.: 10/360,633

(22) Filed: Feb. 10, 2003

Related U.S. Application Data
(63) Continuation-in-part of application No. 10/277,918, filed on Oct. 23, 2002, now abandoned.

(30) Foreign Application Priority Data
Jan. 18, 2002 (JP) 2002-10006

(51) Int. Cl. A61B 5/103

(52) U.S. Cl. 600/593

(57) ABSTRACT

A capsule endoscope body is provided with a spatial frequency characteristic converter which causes the optical transfer function of the imaging system of the capsule endoscope to remain essentially constant within some range of in-focus position. The depth of field of images obtained from the capsule endoscope body is increased by signal processing in order to undue the effects of the spatial frequency characteristic converter. Also, signal processing to reduce variations in image quality due to manufacturing tolerances can be provided within the capsule endoscope body. It is preferred, however, that the signal processing be performed within a receiver which is separate from the capsule endoscope body or, ideally, within a personal computer that receives image data signals from the receiver via cable or wireless communications, processes these image signals, and outputs corrected image signals to a display device.

### CROSS REFERENCE TO RELATED APPLICATION

This application is a continuation-in-part of U.S. application Ser. No. 10/277,918 filed Oct. 23, 2002, entitled “Capsule Endoscope”, now abandoned. This application claims the benefit of priority from the prior Japanese Patent Application No. 2002-010,006, filed Jan. 18, 2002, the contents of which are herein incorporated by reference.

### BACKGROUND OF THE INVENTION

Conventional endoscopes are formed of a first part with a tip portion that is inserted into a patient's body for observation and treatment, and a control unit that is externally provided and connected to the first part. The first part has electrical devices such as an illumination system and an image sensor within the tip portion. The control unit powers these electrical devices via electric wires.

Conventional endoscopes cause significant pain to the patient when they are inserted into the body. For instance, the patient suffers from significant pain when the tip portion passes through the patient's throat. Moreover, the patient continuously suffers from pain while the tip portion is in the body, thus creating a burden to the patient. To reduce the patient's pain, a small, capsule endoscope body 1 as illustrated in FIG. 1 has been proposed. The patient simply swallows the capsule endoscope body 1 and generally experiences little or no pain thereafter during the time that the capsule endoscope body 1 is within the patient's body.

However, the capsule endoscope body 1 has the following problems.

First, it includes batteries which supply the power necessary for making observations within the patient's body; however, the supply of energy is limited. Usually, approximately 30 hours elapses from the time a capsule endoscope body 1 is swallowed until it is discharged from the body. Because of a shortage of power, conventional capsule endoscopes do not allow observations of the entire path within the body. In order to solve this problem, using more batteries or a battery with increased storage capacity has been considered. However, an increase in battery volume causes the required size of the capsule to increase.

Second, illumination sources as used with conventional capsule endoscopes include halogen lamps and LEDs which provide sufficient brightness for observation in narrow, tubular parts of the body such as the esophagus 2, but yield insufficient illumination for observation within larger spaces, such as the stomach 3 and the large intestine 4. To overcome this, larger LEDs 5, such as shown in FIG. 2, are used to ensure a sufficient brightness. However, the diameter D of the capsule must be increased with an increase in the size of the illumination system. In addition, operating larger LEDs requires more power. To overcome this, either more batteries 14 (FIG. 3) or a battery 14′ (FIG. 4) with increased storage capacity are generally needed. As a result, the overall length L of the capsule must be increased in order to accommodate the necessary increase in volume of the battery or batteries. However, an increase in overall length of the capsule impairs the capsule endoscope's advantage of reducing the patient's pain.

One of the requirements for capsule endoscopes is that the objective lens 6 (FIG. 2) has a large depth of field that spans from the exterior surface of the tip portion cover 9 (FIG. 3) to several tens of millimeters in front of it. Generally, an objective optical system with a higher F number will have a greater depth of field. However, using an objective optical system that has a higher F number will generally restrict the light rays passing through the objective optical system, thereby making the image less bright. To counteract this, the illumination source must be made brighter.

Capsule endoscopes carry a relatively weak illumination source, as described above. Therefore, when the objective optical system has a large F number, the image can be so dark that observation and diagnosis of an object become difficult to perform. For this reason, conventional capsule endoscopes do not extend the depth of field of the objective optical system by using an objective optical system having a high F number. Consequently, conventional capsule endoscopes have a small depth of field.

In order to meet the viewing requirements, the objective optical system 7 of a conventional capsule endoscope, as shown in FIG. 3, is designed to focus at a surface several tens of millimeters away from the front end of the objective optical system. The distance d between the tip portion cover 9 and the front end of the objective optical system is adjusted in accordance with the depth of field of the objective optical system so that the near point of the depth of field matches the exterior surface of the tip portion cover 9. This allows an object to be in focus from the tip portion cover to several tens of millimeters away from the tip portion cover. However, the configuration as shown in FIG. 3 increases the distance d between the tip portion cover and the front end of the objective optical system, and thus results in an increase in overall capsule length L.

The objective optical system of a capsule endoscope is also required to be small. The objective optical system in conventional, non-capsule endoscopes is formed of, for example, many lenses and various filters, the latter being for color correction. For a conventional capsule endoscope, color correction ensures a constant color reproduction when used in combination with an illumination system that employs sources having different spectral outputs. Solid-state elements such as CCD and CMOS devices are especially sensitive to infrared wavelengths. Thus, this non-linear sensitivity can result in there being optical noise that is introduced during the imaging process. Therefore, a filter to reduce the intensity of infrared wavelengths is generally positioned within the objective optical system. For this reason, the objective optical system of a conventional, non-capsule endoscope has a large overall length. Because it is formed of many optical elements, the objective optical system of a conventional, non-capsule endoscope suffers from both high cost of components and high cost of assembly. Thus, prior art objective optical systems for non-capsule endoscopes are not appropriate for use as an objective optical system for a capsule endoscope.

A capsule endoscope body may be controlled by magnetic induction in order to affect its location/orientation during the observation period within a patient's body. Therefore, it is important that the capsule endoscope body be lightweight. The capsule endoscope body is also required to be disposable. Therefore, reducing the production cost per capsule is crucial.

To satisfy the above requirements, the objective optical system in capsule endoscopes may be formed using plastic lenses. However, plastic lenses are subject to relatively large changes in their shape, depending on the temperature and amount of hydration of the plastic lenses. Changes in physical characteristics, such as the refractive index, also occur. Thus, the temperature, amount of moisture in the body, and elapsed time since being ingested, greatly affect the imaging performance of the objective optical system. This leads to a problem in that changes in the depth of field of the objective optical system that occur during the course of an observation within a patient's body may result in a failure of the capsule endoscope to provide needed images. To avoid this, the prior art has considered variations in the production tolerances of the objective optical system and variations in the depth of field under actual circumstances, so that focusing is performed with higher precision. This makes assembly more difficult, decreases the yield of acceptable product, and thus results in an increase in the cost of production.

### BRIEF SUMMARY OF THE INVENTION

A first object of the invention is to ensure a large depth of field for a capsule endoscope; a second object of the invention is to extend the time period that observations may be taken using a capsule endoscope; a third object of the invention is to make a capsule endoscope body smaller in size; a fourth object of the invention is to reduce the costs of components within a capsule endoscope body; and a fifth object of the invention is to reduce the costs of assembly of a capsule endoscope body.

### BRIEF DESCRIPTION OF THE DRAWINGS

The present invention will become more fully understood from the detailed description given below and the accompanying drawings, which are given by way of illustration only and thus are not limitative of the present invention, wherein:

FIG. 1 is a schematic diagram of portions of the interior of a human body that may be examined by the present invention;

FIG. 2 is a schematic diagram showing a front view of a capsule endoscope body of the prior art;

FIG. 3 is a schematic diagram that shows the internal structure of the capsule endoscope body shown in FIG. 2;

FIG. 4 is a schematic diagram that shows the internal structure of another capsule endoscope body of the prior art;

FIG. 5 shows the overall configuration of a first example of a capsule endoscope observation system according to the present invention;

FIG. 6 is a schematic diagram that shows the internal structure of a capsule endoscope body according to Embodiment 1 of the present invention;

FIG. 7 is a schematic diagram showing a front view of the capsule endoscope body according to Embodiment 1 of the present invention;

FIG. 8 is a schematic diagram that shows the internal structure of a capsule endoscope body according to Embodiment 2 of the present invention;

FIG. 9 is a schematic diagram that shows the internal structure of a capsule endoscope body according to Embodiment 3 of the present invention;

FIG. 10 is a schematic diagram that shows the internal structure of a capsule endoscope body according to Embodiment 5 of the present invention;

FIG. 11 is a schematic diagram that shows the internal structure of a capsule endoscope body according to Embodiment 6, considered the “best mode”, of the present invention;

FIGS. 12(a) and 12(b) show the detailed structure of the objective optical system of the capsule endoscope of Embodiment 6, which embodiment is currently the “best mode” of the invention, with FIG. 12(a) being a side view and FIG. 12(b) being an end view as seen from the object side;

FIG. 13 shows the detailed structure of another example of an objective optical system that may be used in the capsule endoscope of Embodiment 6;

FIG. 14 shows the internal structure of the capsule endoscope body of Embodiment 7;

FIGS. 15(a) and 15(b) show the detailed structure of the objective optical system of Embodiment 7, with FIG. 15(a) being a side cross-sectional view, and FIG. 15(b) being an end view, as seen from the object side;

FIGS. 16(a) and 16(b) show the detailed structure of another example of the objective optical system of Embodiment 7, with FIG. 16(a) being a side cross-sectional view, and FIG. 16(b) being an end view, as seen from the object side;

FIG. 17 shows the internal structure of the capsule endoscope body of Embodiment 8 of the present invention;

FIG. 18 shows the detailed structure of the objective optical system of Embodiment 8 of the present invention;

FIGS. 19(a) and 19(b) show the detailed structure of the objective optical system of Embodiment 9 of the present invention, with FIG. 19(a) being a side cross-sectional view, and FIG. 19(b) being an end view, as seen from the object side;

FIG. 20 is a schematic front view of the capsule endoscope body of Embodiment 10 of the present invention;

FIG. 21 shows the detailed structure of the objective optical system of Embodiment 10 of the present invention;

FIG. 22 shows an extended depth of field imaging system of the prior art, which heretofore has not been used to extend the depth of field of a capsule endoscope imaging system;

FIG. 23 is a perspective view to show the appearance of the mask, shown in FIG. 22, and which functions as a spatial frequency characteristic converter to cause the optical transfer function of the imaging system of FIG. 22 to remain essentially constant within some range of in-focus position;

FIG. 24 is a graphical presentation to show the intensity profile of the optical transfer function when an object is at the focal point in a general optical system;

FIG. 25 is a graphical presentation to show the intensity profile of the optical transfer function when an object is away from the focal point in a general optical system;

FIG. 26 is a graphical presentation to show the intensity profile of the optical transfer function when an object is farther away from the focal point than in FIG. 25 in a general optical system;

FIG. 27 is a graphical presentation to show the intensity profile of the optical transfer function when an object is at the focal point in an optical system having an extended depth of field;

FIG. 28 is a graphical presentation to show the intensity profile of the optical transfer function in an optical system having an extended depth of field when an object is away from the focal point;

FIG. 29 is a graphical presentation to show the intensity profile of the optical transfer function in an optical system having an extended depth of field when an object is further away from the focal point than in FIG. 28;

FIG. 30 is a graphical presentation to show the characteristic of an inverse filter for processing the intensity profile of the optical transfer function in an optical system having an extended depth of field;

FIG. 31 is a graphical presentation to show the intensity profile of the optical transfer function after the intensity profile of the optical transfer function of FIG. 27 is processed using the inverse filter having the characteristic of FIG. 30;

FIG. 32 is a graphical presentation to show the intensity profile of the optical transfer function after the intensity profile of the optical transfer function of FIG. 28 is processed using the inverse filter having the characteristic of FIG. 30;

FIG. 33 is a graphical presentation to show the intensity profile of the optical transfer function after the intensity profile of the optical transfer function of FIG. 29 is processed using the inverse filter having the characteristic of FIG. 30;

FIG. 34 is a graphical presentation to show the spectral radiance of an LED;

FIG. 35 shows the overall configuration of a second example of a capsule endoscope observation system according to the present invention;

FIG. 36 shows a cross-sectional view of a first example of an objective lens system that may be used in the capsule endoscope of the present invention;

FIGS. 37(a) and 39(b) are graphs of the optical transfer function of the objective lens system shown in FIG. 36, with the object at three different distances and with the image point on-axis versus at the periphery of the image (i.e., at the maximum image height);

FIGS. 40(a)-42(b) are graphs of the optical transfer function of an objective lens system that is identical to that shown in FIG. 36, except that surface #2 is planar rather than aspherical;

FIG. 43 shows a cross-sectional view of a second example of an objective lens system that may be used in the embodiments of a capsule endoscope according to the present invention;

FIGS. 44(a) and 46(b) are graphs of the optical transfer function of a second example of an objective lens system that may be used in the embodiments of a capsule endoscope according to the present invention; and

FIGS. 47(a)-49(b) are graphs of the optical transfer function of the objective lens system shown in FIG. 43, with the object at three different distances and with the image point on-axis versus at the periphery of the image (i.e., at the maximum image height).

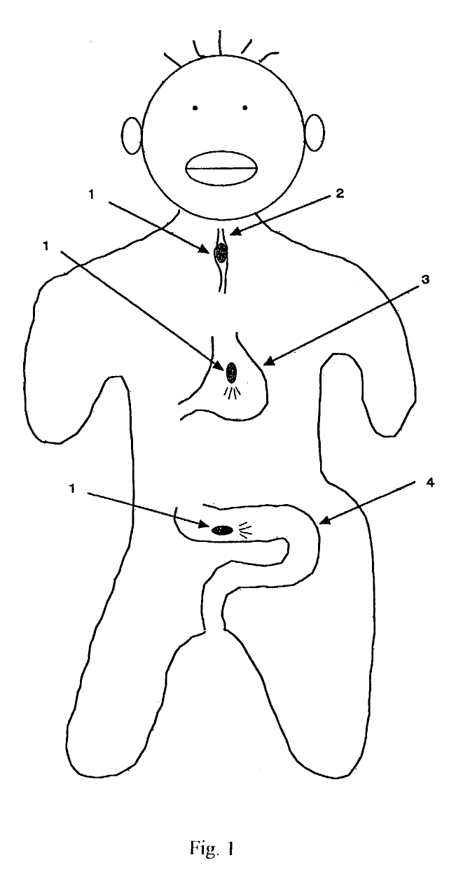

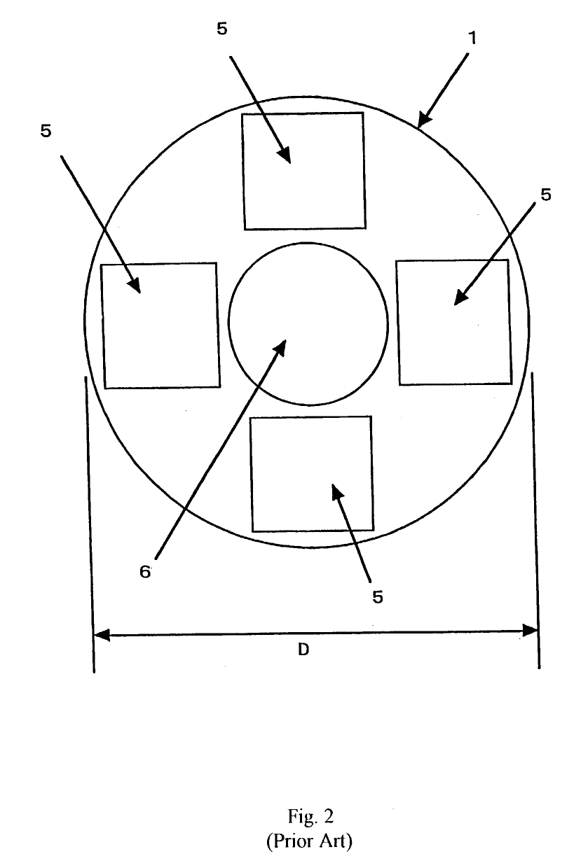

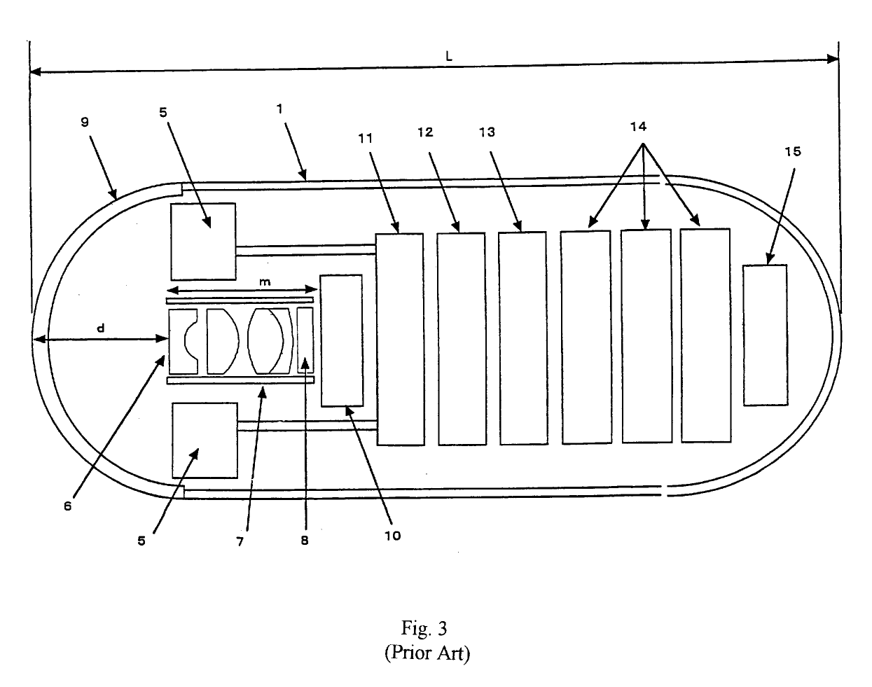

### DETAILED DESCRIPTION

The capsule endoscope according to the present invention has the following characteristics. A capsule endoscope body includes an illumination system for illuminating the interior of a living body, an imaging system for imaging the interior part that is illuminated, and a transmitter for transmitting image signals captured and output by the imaging system, all of which are housed in a sealed capsule.

The imaging system is formed of an objective optical system, a spatial frequency characteristic converter, and an image sensor which scans the images so as to convert the images into electrical output signals. The spatial frequency characteristic converter consists of an optical mask as taught in U.S. Pat. No. 5,748,371, the disclosure of which is hereby incorporated by reference. The spatial frequency characteristic convertor causes the optical transfer function to remain essentially constant within some range of in-focus position. This enables the depth of field of the imaging system to be increased while retaining good image quality.

A signal processor, as also taught in U.S. Pat. No. 5,748,371, is used to restore the spatial frequency property that has been transformed by the spatial frequency characteristic converter. The signal processor may be provided within the capsule endoscope body itself or outside of the capsule endoscope body. Preferable, the signal processor is provided outside of the capsule endoscope body, as will be discussed below.

A receiver for receiving image signals that are transmitted from the capsule endoscope body is provided away from the capsule endoscope body. The receiver preferably includes the signal processor which restores the spatial frequency property that has been transformed by the spatial frequency characteristic converter within the capsule endoscope body to thereby produce high-resolution images of the interior part over an extended depth of field. The image sensor is a MOS type image sensor. As in prior art capsule endoscopes, the power source for operating the capsule endoscope may include one or more batteries.

According to the present invention, a power source for at least partially powering the capsule endoscope body may be provided outside of the capsule endoscope body. For example, the capsule endoscope body may be powered in whole or in part by electromagnetic waves, such as microwaves, which are transmitted from an external power source. The objective optical system may be formed of plural plastic lenses. The sealed capsule has a transparent tip portion cover that covers the front of the objective optical system and an illumination system. Furthermore, the tip portion cover may have a substantially oval shape.

Alternatively, the tip portion cover may have a shape at the part that covers the front of the objective optical system that is different from the part that covers the front of the illumination system. Furthermore, a member that provides a shielding effect may be provided between the objective optical system and the illumination system. The spatial frequency characteristic converter preferably has an aperture which is analogous in shape to that of the light receiving part of the image sensor, which is generally rectangular in shape. The lens frame for supporting the objective optical system has an aperture on the object side that also serves as a brightness stop. The shape of the brightness stop is also preferably the same as that of the spatial frequency characteristic converter. The objective optical system may be formed of a single aspherical lens of positive refractive power, a lens group having positive refractive power which consists of two positive lenses, or a first lens group having an overall negative refractive power and a second lens group having an overall positive refractive power.

A lens frame for supporting the objective optical system has an opening on the object side that serves as a brightness stop, with the spatial frequency characteristic converter being positioned at substantially the same position as the brightness stop of the objective optical system.

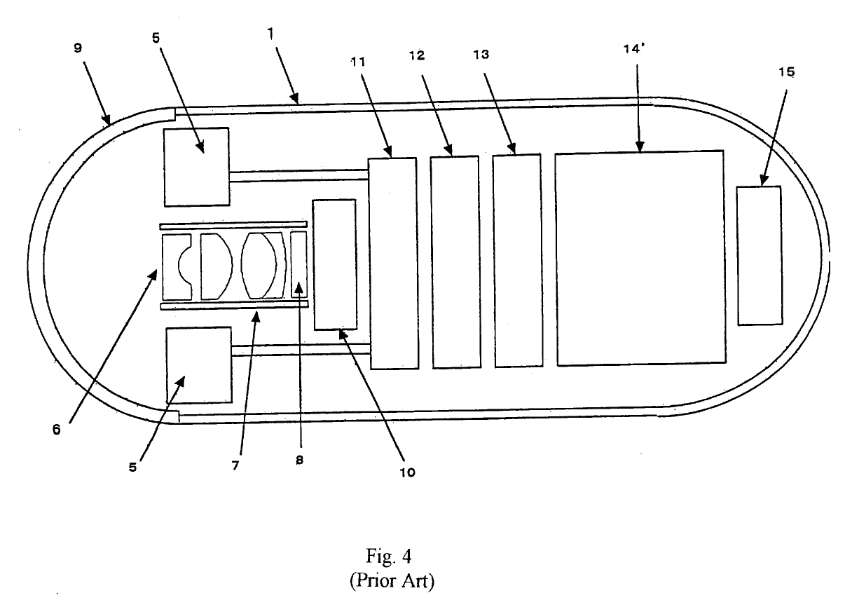

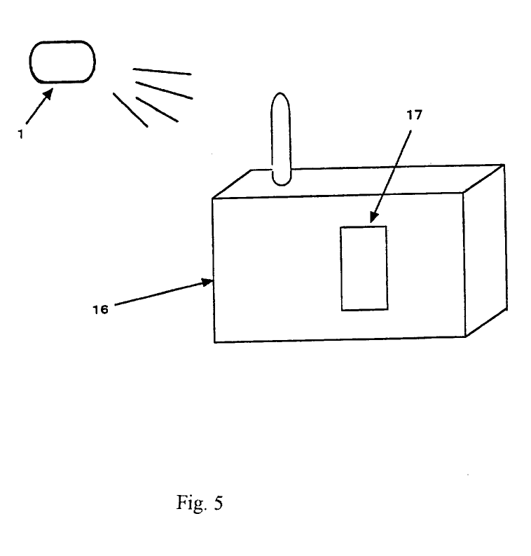

FIG. 5 shows a first example of an overall configuration of a capsule endoscope observation system according to the present invention, comprising a capsule endoscope body 1 and a receiver unit 16 that is set up nearby to receive images from the capsule endoscope body so that the received images may be displayed on a monitor (not shown).

Various embodiments of the invention will now be set forth in detail.

### Embodiment 1

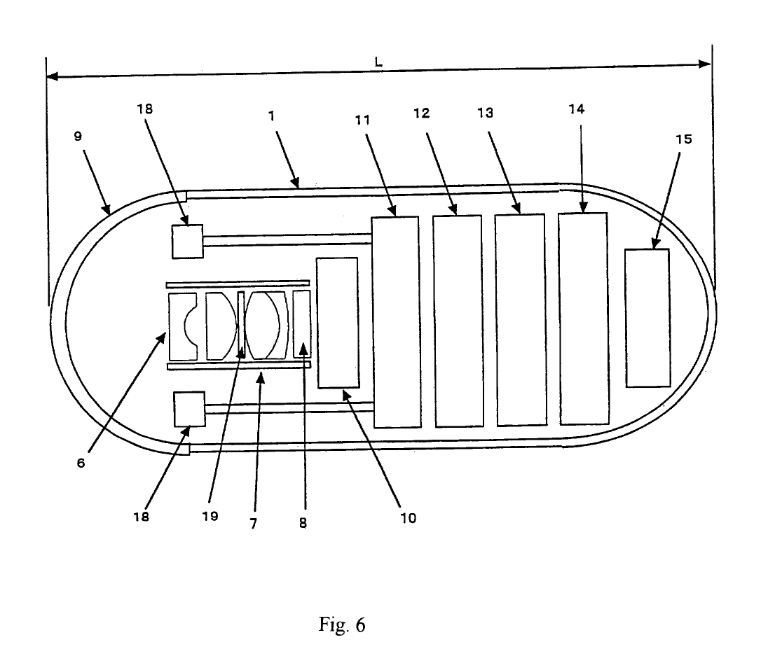

FIG. 6 shows the structure of a capsule endoscope body 1 according to Embodiment 1 of the present invention. The capsule endoscope body is formed of a transparent cover 9 for enclosing the capsule endoscope body 1, an illumination system 18, an objective optical system 7 including an objective lens 6, an infra-red blocking filter 8, a solid-state imaging element 10, an image processing unit 12 for controlling the solid-state imaging element 10 and processing images, a control unit 11, a wireless unit 13, an antenna 15, and a power unit 14. This embodiment uses batteries for the power unit 14.

An objective optical system 7 is provided with a spatial frequency characteristic converter 19 for transforming the spatial frequencies. Also, a signal processing circuit is included in an image processing unit 12 that is provided within the endoscope capsule body. Before shipping of the capsule endoscope body, the image processing unit 12 is used to adjust for variations in optical performance due to production tolerances of the objective optical system 7. This enables the capsule endoscope system to have a constant imaging performance despite differences in optical performance among the individual capsule endoscope bodies, and also improves the production yield by reducing the number of capsule endoscope bodies that must be rejected for quality control reasons. The signal processing circuit is used to restore, using digital processing, the spatial frequency content of image signals so as to remove variations in the spatial content of image signals due merely to production tolerances of optical systems of individual capsule endoscope bodies.

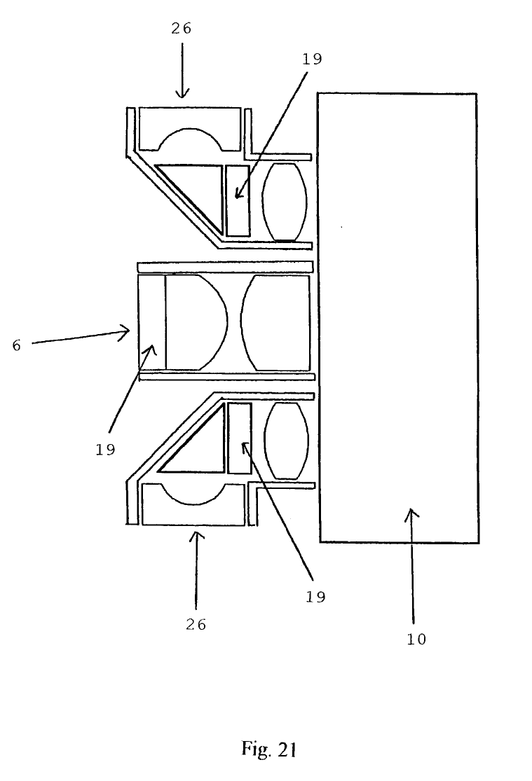

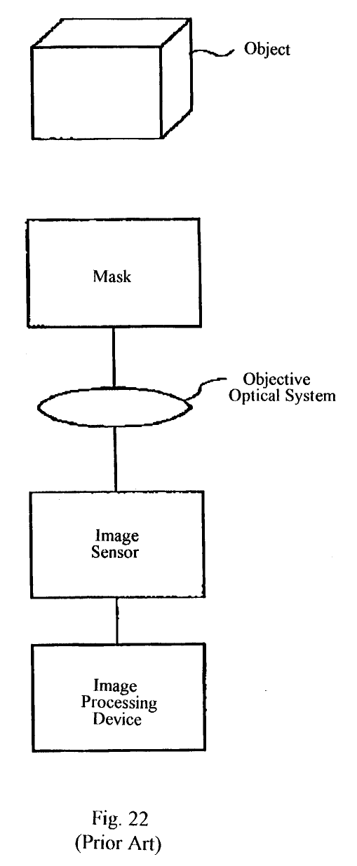

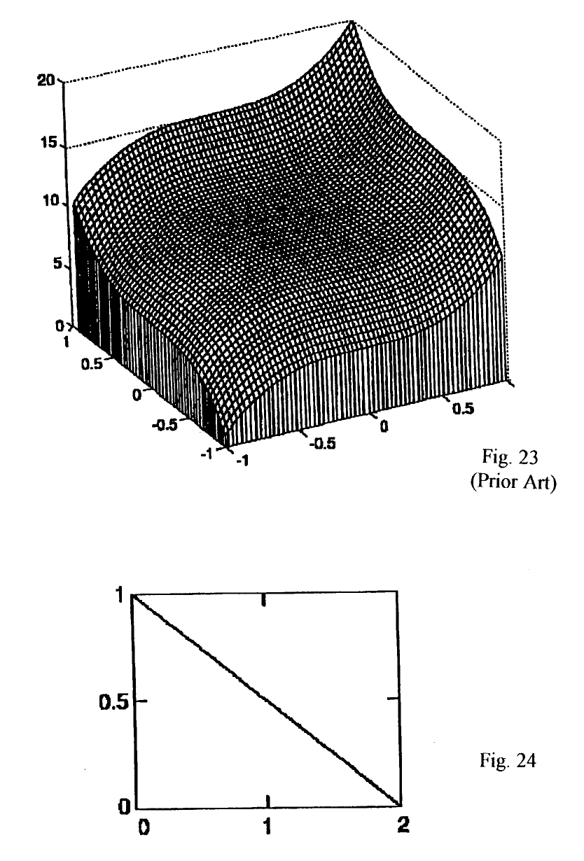

As mentioned above, an extended depth of field optical system is disclosed in U.S. Pat. No. 5,748,371. FIG. 22 is a schematic illustration of such a prior art optical system which heretofore has not been used in conjunction with the imaging system of a capsule endoscope. The extended depth of field optical system is formed of an objective optical system (such as the positive lens illustrated) which forms an image of an object by observing the object through a mask. The mask is positioned at the pupil position of the objective optical system, and preferably is a transparent phase mask having a shape as illustrated in FIG. 23. The mask affects the optical transfer function of the optical system by delaying certain spatial frequencies of light from the object more than other spatial frequencies. An image sensor, such as the CCD array illustrated in FIG. 22, captures the modified image data. An image processing device, such as the digital processing system illustrated, is used to undo the effects of the mask, to thereby enable images with an extended depth of field to be displayed for viewing.

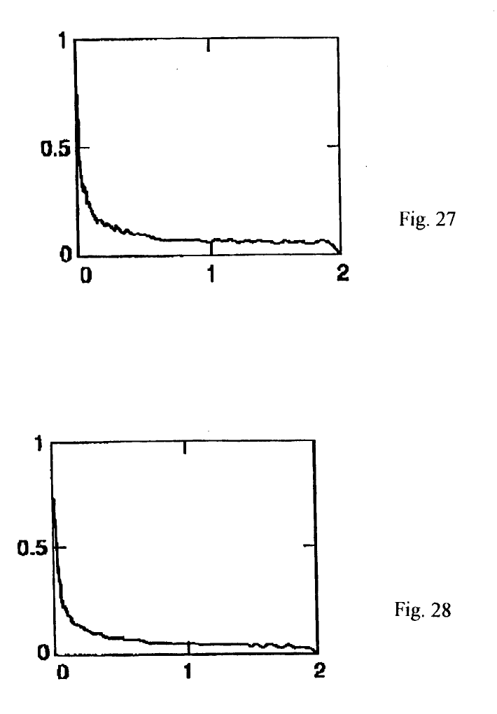

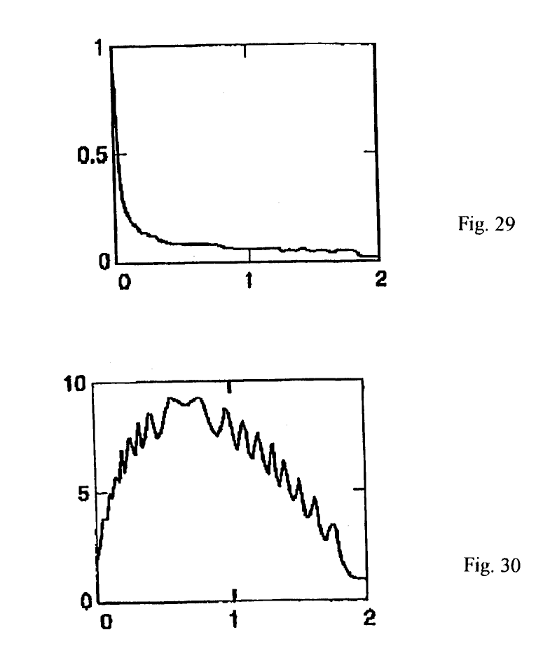

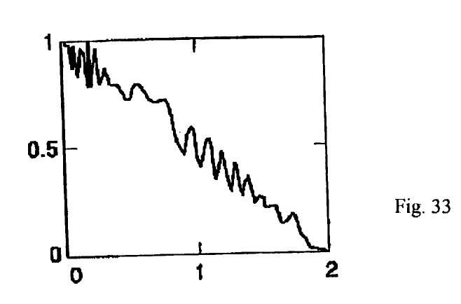

FIGS. 24-33 are graphs of the normalized percent transmission (plotted on the Y-axis) versus spatial frequency at the image plane in line pairs per mm (plotted on the X-axis). Such graphs are commonly referred to as the optical transfer function or OTF. In these figures, the number “2” on the X-axis corresponds to the Nyquist frequency for the imaging element.

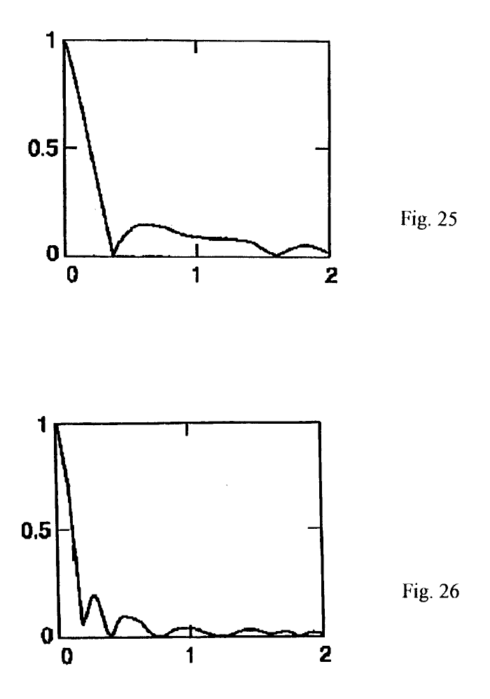

For a conventional optical imaging system without a phase mask, the optical transfer function is as shown in FIG. 24 when an object is positioned at the focal point of the optical system. When the object is moved a given distance away from the focal point of the optical system, the optical transfer function degrades from that shown in FIG. 24 to that shown in FIG. 25. If the object is moved still farther from the focal point of the optical system, the optical transmission function degrades still further as shown in FIG. 26.

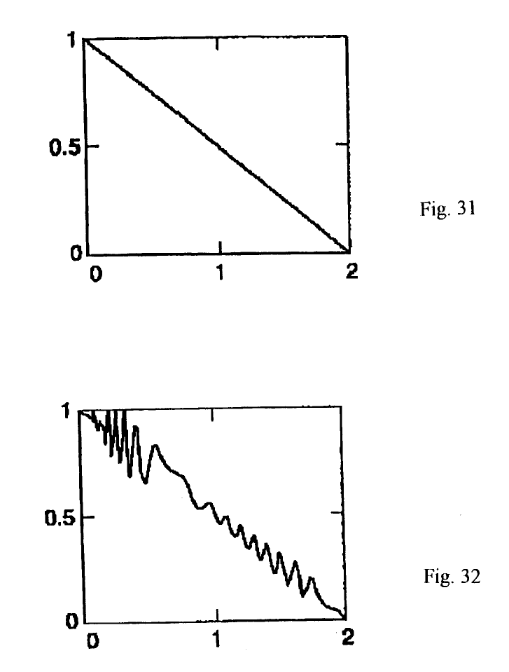

On the other hand, using an optical system having the same optical performance but with an optical phase mask as shown in FIG. 23 (which functions as a spatial frequency characteristic converter) results in the optical transfer functions being as illustrated in FIGS. 27, 28, and 29, for the same respective positions of the object relative to the focal point of the optical system. If a filtering process is performed for the intensity profiles shown in FIGS. 27, 28, and 29 using an inverse filter having a characteristic as shown in FIG. 30, the OTF profiles become as shown in FIGS. 31, 32, and 33, respectively, which are similar to the OTF profiles on the image plane when the object is at the focal point.

As mentioned above, the spatial frequency characteristic converter that is used in the capsule endoscope body of the present invention causes the optical transfer function of the imaging system to remain substantially unchanged within some range of in-focus position, as illustrated by the nearly flat regions of the curves illustrated in FIGS. 27, 28 and 29. The spatial frequency characteristic converter 19 may be formed substantially at a pupil of the objective optical system 7, as shown in FIG. 6. Signal processing is then performed to restore the spatial frequency of image signals obtained from a solid-state imaging element 10 that is positioned at the image plane of the optical system. This can overcome the problems with conventional capsule endoscopes and provide an imaging optical system with a greatly increased depth of field.

Thus, the objective optical system 7 can have a small F number and simultaneously provide a large depth of field so as to ensure that bright images can be formed on the solid-state imaging element 10. This allows a capsule endoscope body to obtain images of the interior parts of larger spaces within a living body, such as required when the capsule endoscope is within the stomach and large intestine.

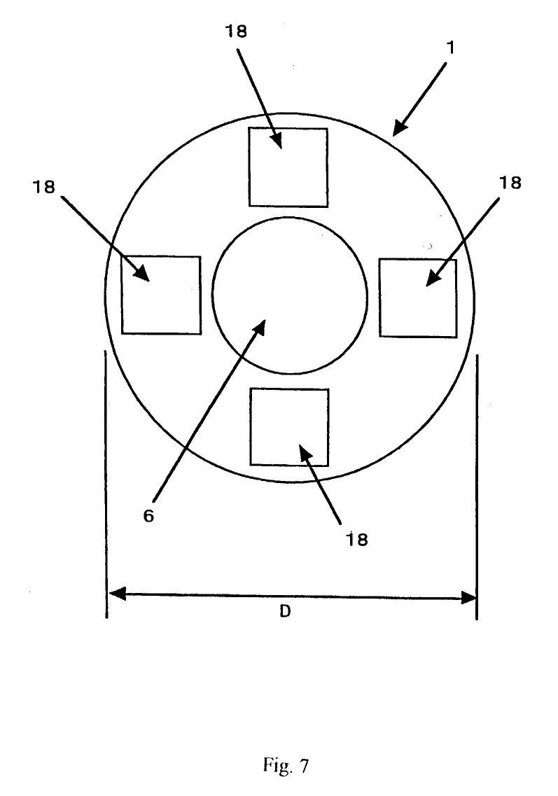

Enabling the objective optical system to have a small F number allows the illumination system 18 to be designed as a low power LED, which leads to downsizing of the illumination system and reducing its power consumption. More advantageously, the capsule endoscope body 1 can now be designed to have a smaller diameter D as is shown in FIG. 7, reducing the patient's pain. Surplus power as a result of using a smaller LED that requires less power allows an extended time for observation and diagnosis by the capsule endoscope body when within a patient. The battery 14 (FIG. 6) can have a significantly reduced capacity and, therefore, a reduced volume. This can shorten the overall length L of the capsule 1 as is shown in FIG. 6.

The extended depth of field that can be provided by the objective optical system 7 is considerable. Therefore, a sufficient depth of field can be achieved for observation and diagnosis of an object without requiring any focusing operation. This simplifies the assembly of the capsule endoscope body 1. In addition, whereas plastic lenses previously would sometimes result in focusing failures due to the optical properties of plastic being more dependent on temperature and humidity, a lack of the need for focusing as in the present invention enables plastic lenses to be used. Therefore, improved yield as well as decreased cost of manufacture and assembly of the objective optical system within the capsule endoscope body should result.

The objective optical system of this embodiment has a wide field of view of approximately 140° that is obtained using a retro-focus-type lens arrangement that is formed of, in order from the object side, a lens group of negative refractive power that is formed of a negative lens element and a positive lens element, a brightness stop with a spatial frequency characteristic converter positioned at the brightness stop, and a lens group of positive refractive power that is formed of a positive lens element that is joined to a negative lens element. Because of the difficulty in orienting a capsule endoscope body within a patient, a wide-angle field of view objective optical system combined with the extended depth of field as available in the present invention is extremely useful in insuring that larger cavities within a living body are properly observed by the capsule endoscope. A sanded diffusing plate or a concave lens which results in a broadly diverging illumination beam can be placed in front of the light exit surface of the LED in order to supplement any shortage of light in the peripheral areas of the expanded field of view. In this way, an optical illumination system with a broad distribution of light is provided, and aids in ensuring that larger cavities within a living body are properly observed by the capsule endoscope. This embodiment includes, within the capsule endoscope body 1, an image processing unit 12, whose function is as discussed above.

It is useful for wireless capsule endoscopes, in order to save power, to use an image compression technique such as a JPEG format before transmitting the image data. The JPEG format affects the spatial frequency content of images that have been compressed using this format by omitting high spatial frequencies from the image data. The present embodiment's design enables image compression to occur with a fewer number of circuits, thus reducing the production cost. Furthermore, the spatial frequency property restoration and image compression are controlled for each capsule endoscope body, ensuring precise image reproduction without variations in the image quality due to production tolerances. Moreover, the spatial frequency property restoration is performed before the image compression, in order to minimize loss in image quality due to the JPEG format irreversibly omitting higher spatial frequencies. In this manner the quality of images obtained using the capsule endoscope is improved.

### Embodiment 2

FIG. 8 shows the structure of the capsule endoscope body 1 of Embodiment 2, which is different from Embodiment 1 in that a tip portion of transparent cover 9 for covering the illumination and objective optical systems has a substantially oval shape. As described above, the depth of field of the imaging system of the capsule endoscope body is increased using the present invention. Therefore, even with a small distance d between the observation point and the first lens surface, focusing is achieved at the point that is in contact with the tip portion cover 9. Thus, the overall length L of the capsule body can be further reduced without degrading observations made with the capsule endoscope. This enables the tip portion of the cover to be formed of inexpensive, molded plastic having any shape. In this embodiment, an oval shape is used.

### Embodiment 3

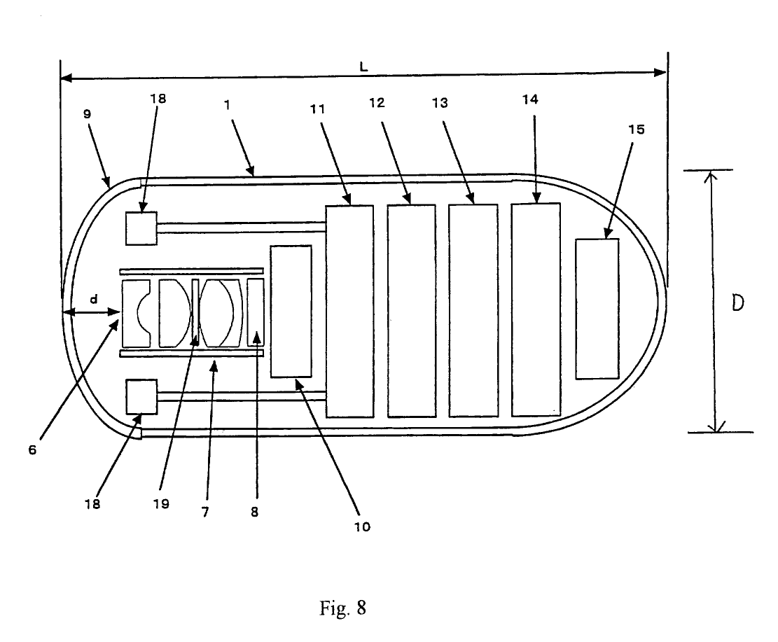

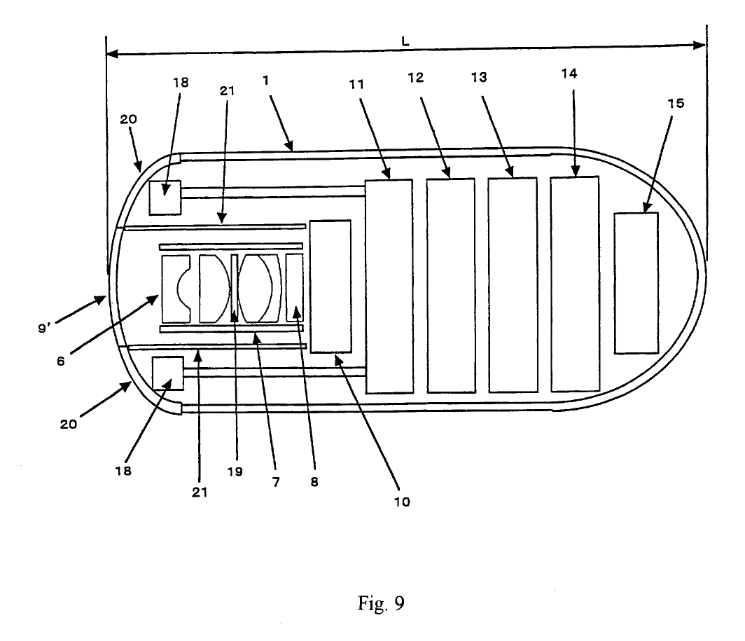

FIG. 9 shows the structure of the capsule endoscope body 1 of Embodiment 3, which is different from Embodiments 1 and 2 in the structure of the tip portion of transparent cover 9 and the presence of a shielding member 21 between the objective optical system 6 and the illumination system 18. The tip portion cover of this embodiment is formed of different materials for the part 9′ that covers the objective optical system and for the part 20 that covers the illumination system.

In the structure of Embodiment 1, a single tip portion covers the objective optical system 7, and the illumination system 18 allows light exiting from the optical illumination system 18 to reflect at the tip portion of the transparent cover 9. This produces stray light, which then enters the objective optical system 7 and easily causes a flare in the field of view. A positional adjustment among the objective optical system, the illumination system 18, and the tip portion of the transparent cover 9 is often made in order to prevent entry of any stray light into the objective optical system. However, such an adjustment makes assembly of the capsule endoscope body 1 more difficult. The structure of this embodiment can easily prevent stray light from entering the objective optical system and, therefore, obviate the need for a positional adjustment among the objective optical system 7, the illumination system 18, and the tip portion of the cover. Therefore, the capsule endoscope body 1 is easier to assemble, enabling an increased yield to be achieved and reducing production cost.

### Embodiment 4

Embodiment 4 is different from Embodiment 1 in that it includes, in the receiver unit 16 of FIG. 5, a signal processing circuit 17 (for restoring the spatial frequency property so as to form images with an extended depth of field). Providing the signal processing circuit 17 in the receiver unit 16 instead of within the capsule endoscope body 1 can simplify the signal processing and circuit structure that is required within the capsule endoscope body 1. This allows further power savings and an extended time for observation and diagnosis of the capsule endoscope body within a patient, realizing a more practical capsule endoscope system. Furthermore, batteries having a smaller storage capacity and smaller volume can be used, allowing a further downsizing of the capsule. When irreversible compression such as JPEG format is used, the high spatial frequency components of an image are eliminated. Therefore, the receiver unit 16 receives image signals with reduced high spatial frequency content which varies according to the image compression ratio. The signal processing circuit 17, in the receiver unit 16, for restoring the spatial frequency property, can perform an optimized process that is targeted for the medium to low frequency property of the image signals. This can simplify the signal processing circuit 17, reducing the production cost of the capsule endoscope system. Generally, high frequency components for the image signals include noise from electrical elements such as the image sensors, and this noise can be amplified during image reproduction by the image processing circuit. This embodiment achieves a reproduced image with less noise by using signal processing that emphasizes the medium to low spatial frequency components and de-emphasizes the high spatial frequency components.

### Embodiment 5

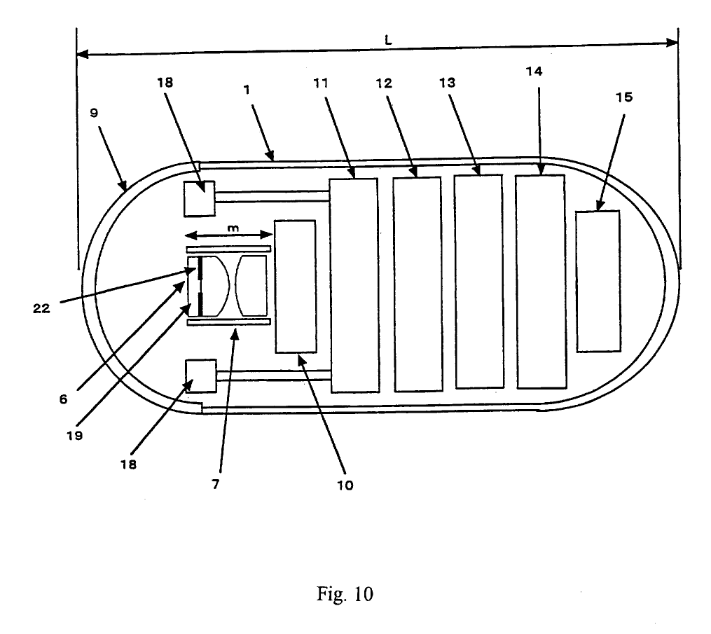

FIG. 10 shows the structure of the capsule endoscope body 1 of Embodiment 5, which is different from Embodiment 1 with regard to the structure of the objective optical system. The objective optical system of this embodiment includes a spatial frequency characteristic converter 19. The objective lens 6 is formed of two positive lens elements. Accordingly, the objective optical system is formed of, in order from the object side, the spatial frequency characteristic converter 19, a brightness stop 22, two plano-convex lenses, and the light receiving surface of a solid-state imaging element 10. Generally, an objective optical system formed of only positive lenses makes for a compact imaging unit but does not provide a sufficiently large back focus. The objective optical system 7 of this embodiment has the spatial frequency characteristic converter 19 very near the brightness stop 22, thereby reducing the overall length m of the objective optical system 7. This can further reduce the overall length L of the capsule endoscope body.

The objective optical system 7 of this embodiment does not include an infrared filter or color correction filter. As described in the prior art, conventional objective optical systems for endoscopes require an infrared filter or color correction filter. However, the capsule endoscope includes the illumination unit and imaging unit together within the capsule. Therefore, color reproduction for the imaging unit is determined according to the spectral intensity property of the illumination unit. For this reason, there is no need for a color correction filter in the objective optical system. In addition, a white LED is used as the illumination system. The white LED uses fluorescent substances to create desired colors and, therefore, does not produce significant amounts of ultraviolet or infrared light which may degrade observations using electronic image sensors.

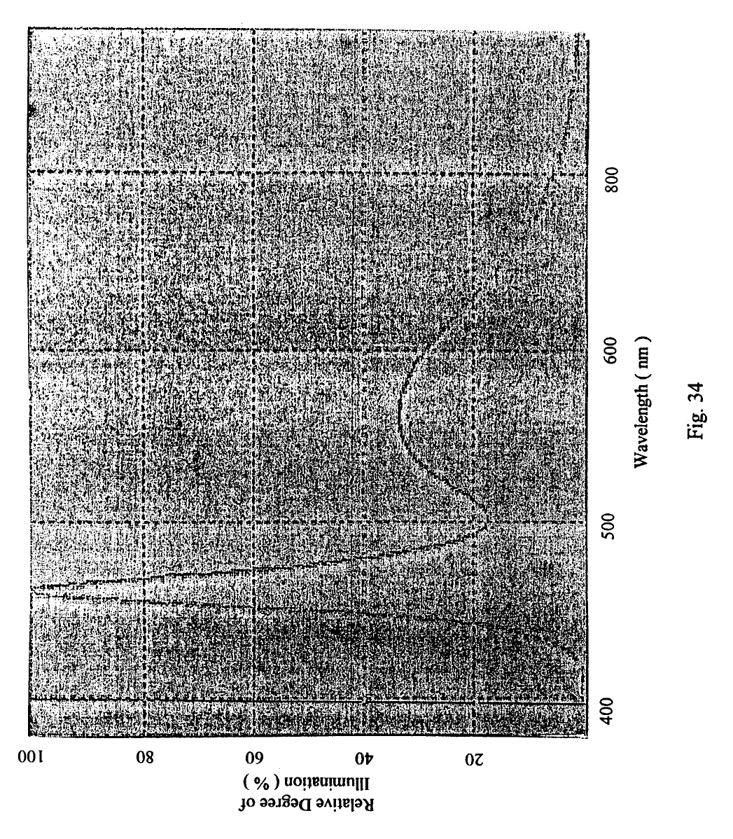

FIG. 34 shows the relative degree of spectral radiance (expressed as a percentage on the Y-axis) versus wavelength of emitted light (expressed in nm on the X-axis) of a white LED. As there are negligible ultraviolet wavelengths and almost no infrared wavelengths generated by the white LED, there is no need to include an infrared blocking filter or an ultraviolet blocking filter in the objective optical system. By eliminating the need for both infrared and color correction filters, a positive refractive power optical system that does not need a large back focus may be used.

### Embodiment 6

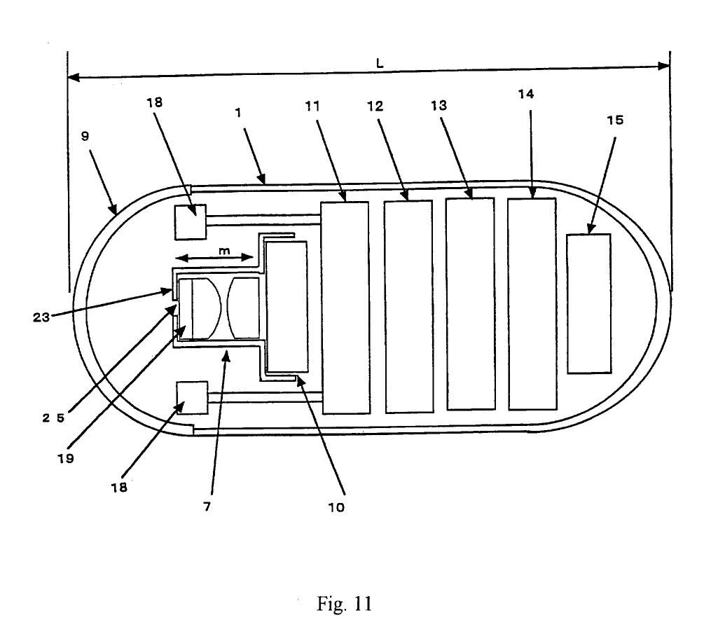

FIG. 11 shows the structure of the capsule endoscope body 1 of Embodiment 6, which is different from Embodiment 5 with regard to the structure of the objective optical system. The objective optical system 7 of this embodiment includes a spatial frequency characteristic converter 19. The objective lens system is formed of two positive lenses. Accordingly, the objective optical system is formed of, in order from the object side, a frame 23 that has an opening on the object side which serves as a brightness stop, the spatial frequency characteristic converter 19, two plano-convex lenses, and the light receiving surface of a solid-state imaging element 10. A frame 23 has an aperture 25 on the object side that serves as a brightness stop, which is immediately followed by the spatial frequency characteristic converter 19. Positioning the spatial frequency characteristic converter 19 immediately after the brightness stop allows the objective optical system to be formed of only two positive lens elements.

The structure includes a capsule endoscope body that houses an illumination means 18, an objective optical unit 7, a solid-state imaging element 10, an image processing unit 12 for controlling the solid-state imaging element 10 and processing images, a total control unit 11, a wireless unit 13, an antenna 15, and a power unit 14, which are sealed within a capsule cover 1 and a transparent cover 9 as is shown in FIG. 11. Also, a receiver 16 is provided with a signal processing circuit 17 for reproducing the spatial frequency properties as shown in FIG. 5. The power unit 14 is a battery which supplies all the power necessary for the capsule endoscope. The solid-state imaging element 10 either a CCD or an MOS type image sensor.

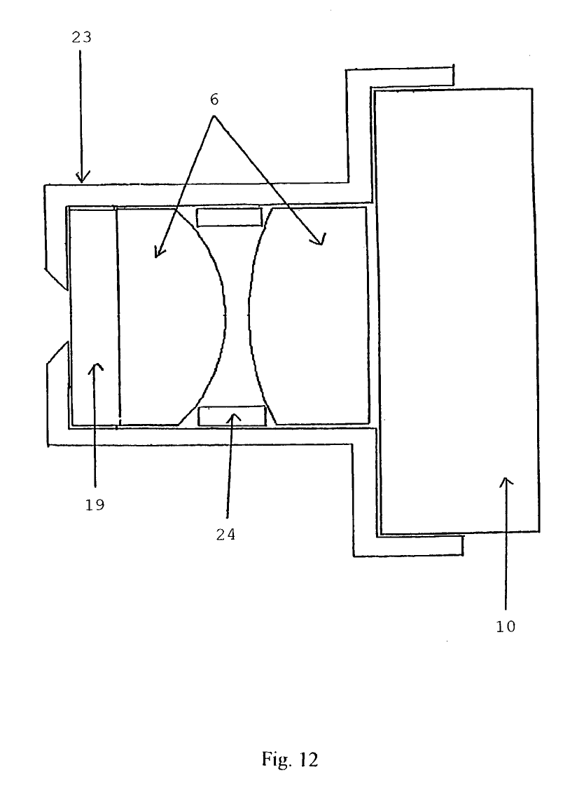

FIGS. 12(a) and 12(b) show the detailed structure of the imaging unit. FIG. 12(a) is a side view and FIG. 12(b) is an end view as seen from the object side. Referring to FIG. 12(a) a spatial frequency characteristic converter 19 that serves as pupil modulating element, a plano-convex lens 6, a ring 24 for spacing, a plano-convex lens 6, and a solid-state imaging element 10 are provided, in order from the object side, in a lens frame 23 having an aperture 25. Referring to FIG. 12(b), the aperture 25 of the lens frame 23 is a brightness stop and has a round shape. The spatial frequency characteristic converter 19 that serves as a pupil modulating element has a circular periphery around the optical axis, as does the plano-convex lens 6. The pupil modulating element 19 has an outer diameter equal to that of the plano-convex lens 6.

As is shown in FIG. 13, the number of parts is further reduced by combining the spatial frequency characteristic converter 19 and the plano-convex lens 6 into a single lens or bonding the oppositely oriented plano-convex lens 6 to the solid-state imaging element 10 in order to increase the assembly performance of the imaging unit. This is also useful to prevent the distances between parts that form the imaging unit from diverging so that the quality of the imaging is stable, while improving the yield.

### Embodiment 7

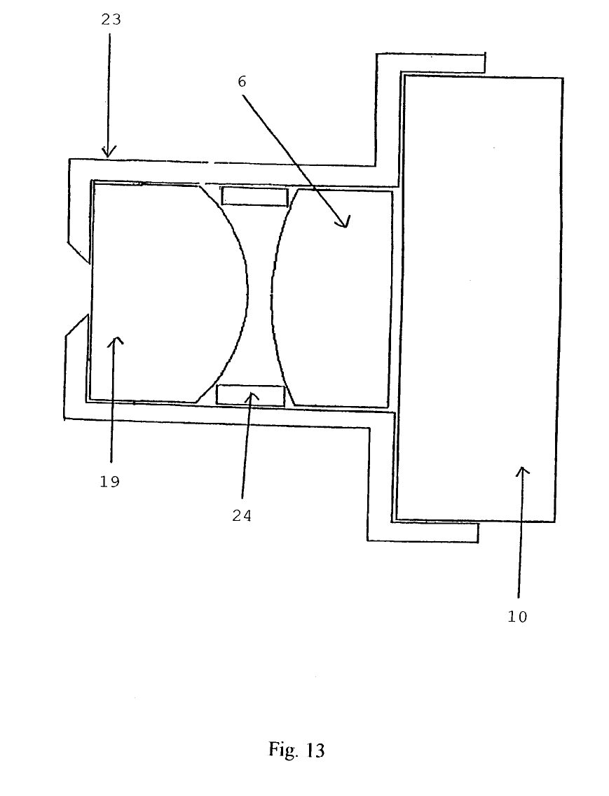

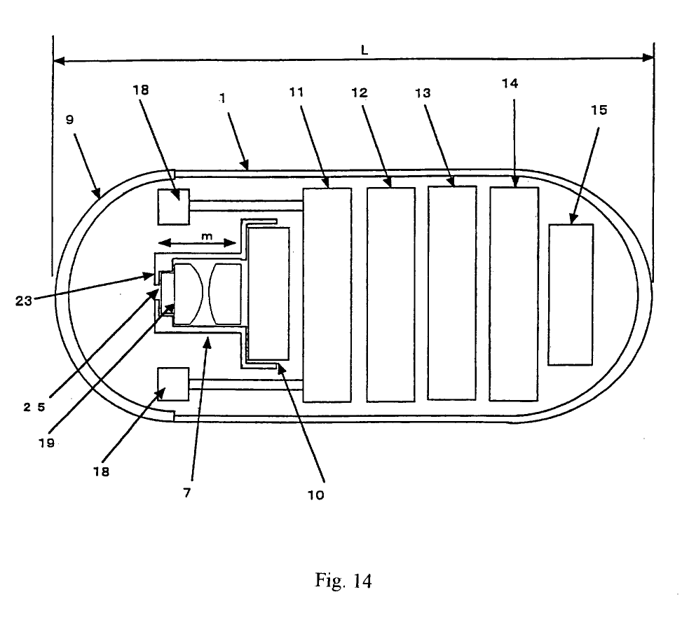

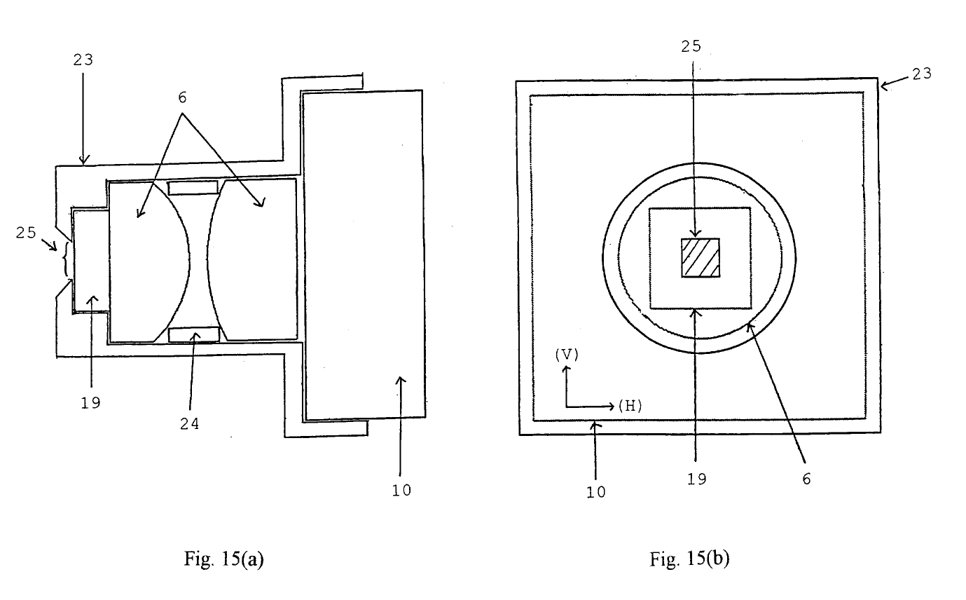

FIG. 14 shows the structure of the capsule endoscope body 1 of Embodiment 7, which is different from Embodiment 6 with regard to the structure of the objective optical system. FIGS. 15(a) and 15(b) are detailed views of the imaging unit of Embodiment 7, with FIG. 15(a) being a side view and FIG. 15(b) being an end view as seen from the object side. The objective optical system 7 includes a spatial frequency characteristic converter 19. The objective lens consists of two plano-convex lenses oriented with their convex sides facing one another, as indicated. Accordingly, the objective optical system consists of, in order from the object side, the spatial frequency characteristic converter 19, the two piano-convex lenses, and the light receiving surface of a solid-state imaging element 10. A frame 23 has an aperture 25 on the object side that serves as a brightness stop, which is immediately followed by the spatial frequency characteristic converter 19.

Referring to FIGS. 15(a) and 15(b), the brightness stop or aperture 25 formed in the frame 23 is substantially a square. The spatial frequency characteristic converter 19 is also substantially a square. Accordingly, the frame 23 has a substantially square inner contour to house the spatial frequency characteristic converter 19. The solid-state imaging element 10 is also formed to be substantially square. Accordingly, the frame 23 has a substantially square inner contour to house the solid-state imaging element 10. A substantially square area of the spatial frequency characteristic converter 19 that faces the brightness stop or aperture 25 has a three-dimensional, curved surface shown in FIG. 23. The vertical (V) and horizontal (H) directions of the pixel array of the solid-state imaging element 10 are aligned with the vertical and horizontal directions of the substantially square area of the spatial frequency characteristic converter 19 and the brightness stop or aperture 25. This can maximize the spatial frequency property transforming performance in the imaging unit for the solid-state imaging element 10. This also contributes to optimization of the spatial frequency property transforming performance in the imaging unit, taking into account the vertical (V) and horizontal (H) resolutions of a monitor.

Using the frame structure of this embodiment eliminates the vertical and horizontal aligning operation of the solid-state imaging element 10, the spatial frequency characteristic converter 19, and the brightness stop or aperture 25. Also, as with Embodiment 6, the parts forming the imaging unit can be inserted and fixed in the frame 23 for assembly. This facilitates assembly by dramatically reducing the labor and time required for assembly. The number of parts can be reduced by molding the spatial frequency characteristic converter 19 to the plano-convex lens that immediately follows it, further improving assembly performance.

FIGS. 16(a) and 16(b) show a modified embodiment of Embodiment 7, with FIG. 16(a) being a side view and FIG. 16(b) being an end view as seen from the object side. The imaging unit includes a spatial frequency characteristic converter and a plano-convex lens that are combined into a single lens 19′ and another plano-convex lens 6, as illustrated. The frame 23 has a structure that determines the distance n between the spatial frequency characteristic converter (i.e., lens 19′) and the plano-convex lens 6 so that the clearance ring between the spatial frequency characteristic converter and the plano-convex lens 6 is eliminated, thus reducing the number of parts. Also in this embodiment, the plano-convex lens 6 and the solid-state imaging element 10 can be bonded together to reduce the number of parts, further improving assembly performance of the imaging unit.

### Embodiment 8

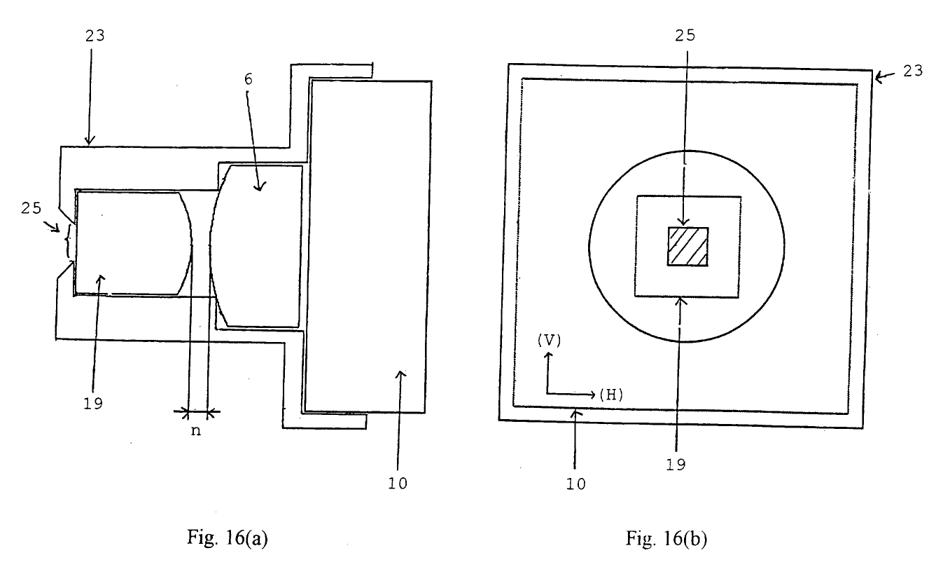

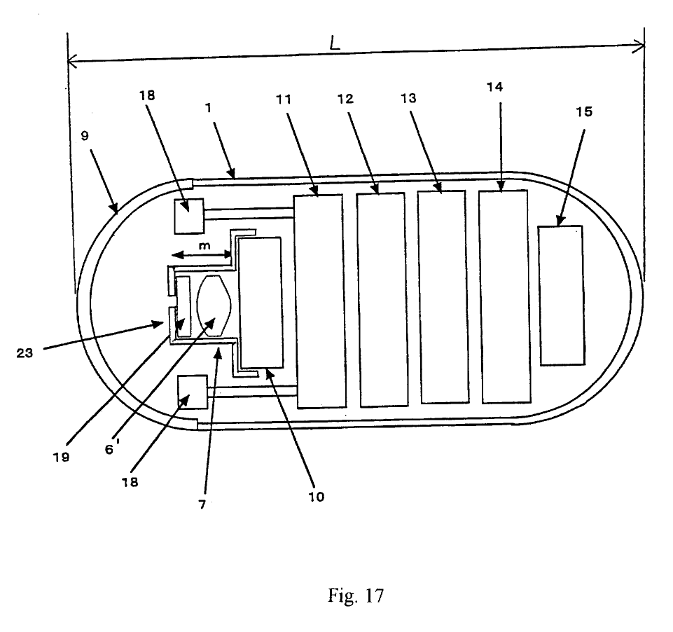

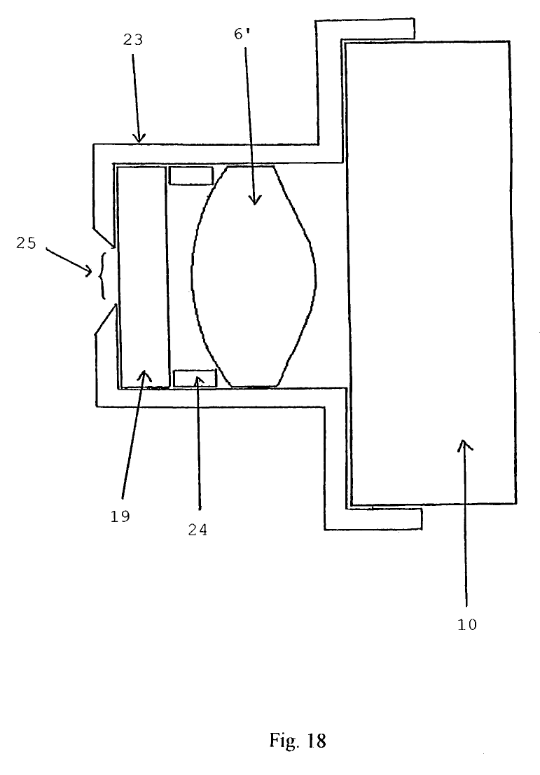

FIG. 17 shows the structure of the capsule endoscope body of Embodiment 8, which is different from that of Embodiments 5, 6, and 7 in the structure of the objective optical system. FIG. 18 is a detailed view of the imaging unit of Embodiment 8. The objective optical system includes a spatial frequency characteristic converter 19, as illustrated element. The objective lens consists of a single, aspherical biconvex lens 6′. Accordingly, the imaging unit consists of, in the following order from the object side, the spatial frequency characteristic converter 19, the aspherical biconvex lens 6′, and the light receiving surface of a solid-state imaging element 10. A frame 23 has an aperture 25 on the object side that serves as a brightness stop, which is immediately followed by the spatial frequency characteristic converter 19.

The structure consisting of a single aspherical biconvex lens can not have a sufficiently large back focus. Therefore, the spatial frequency characteristic converter 19 is positioned at the position of the brightness stop of the objective optical system. An aspherical lens is capable of correcting the field curvature and spherical aberration on its own. This can result in an extremely compact imaging unit with a large depth of field and satisfactorily corrected aberrations while having a wide field angle. Using this imaging unit contributes to the further reduction in the overall length L of the capsule endoscope body. Also, as with Embodiment 6, the parts forming the imaging unit can be inserted and fixed in the frame 23, in order, for assembly. In other words, the spatial frequency characteristic converter 19, clearance ring 24, aspherical biconvex lens 6, and solid-state imaging element 10 are inserted and fixed in the frame 23 in order from the object side. This reduces the cost of assembly of the imaging unit.

### Embodiment 9

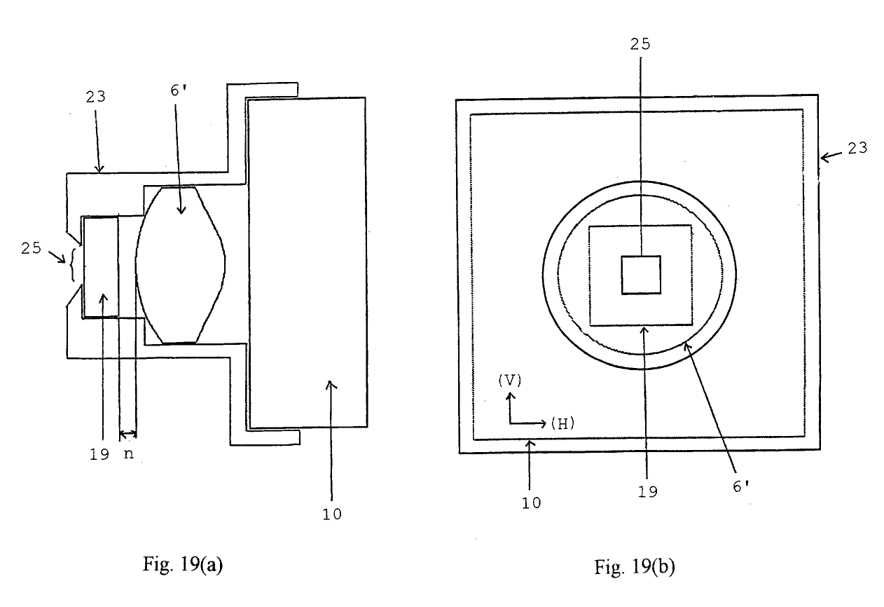

FIGS. 19(a) and 19(b) are detailed views of the imaging unit of Embodiment 9, with FIG. 19(a) being a side view and FIG. 19(b) being an end view, as seen from the object side. Embodiment 9 is different from Embodiment 8 with regard to the structure of the objective optical system. The objective optical system includes a spatial frequency characteristic converter 19. The objective lens consists of a single aspherical lens of positive refractive power. Accordingly, the imaging unit is formed of, in order from the object side, a spatial frequency characteristic converter 19, an aspherical biconvex lens 6′, and the light receiving surface of a solid-state imaging element 10. A frame 23 has an aperture 25 on the object side that serves as a brightness stop, which is immediately followed by a spatial frequency characteristic converter 19. The brightness stop or aperture 25 formed in the frame 23 is substantially a square. The spatial frequency characteristic converter 19 is also formed so as to be substantially square. Accordingly, the frame 23 has a substantially square-shaped inner contour so as to house the spatial frequency characteristic converter 19.

The solid-state imaging element 10 is formed to be substantially square. Accordingly, the frame 23 has a substantially square-shaped inner contour in order to house the solid-state imaging element 10. The substantially square-shaped area of the spatial frequency characteristic converter 19 that faces the brightness stop or aperture 25 has a three-dimensional, curved surface as shown in FIG. 23. The vertical (V) and horizontal (H) directions of the pixel array of the solid-state imaging element 10 are aligned with the vertical and horizontal directions of the substantially square-shaped area of the spatial frequency characteristic converter 19 and the brightness stop or aperture 25. This can enhance the imaging performance of the solid-state imaging element 10.

The frame 23 has a structure that determines the distance n between the spatial frequency characteristic converter 19 and the aspherical biconvex lens 6′. This eliminates the need for a clearance ring between the spatial frequency characteristic converter 19 and the aspherical biconvex lens 6′, further reducing the number of parts. Using the frame structure of this embodiment eliminates the vertical and horizontal aligning operation as in Embodiment 8, namely, of the solid-state imaging element 10, the spatial frequency characteristic converter 19, and the brightness stop or aperture 25. As with Embodiment 8, the parts forming the imaging unit can be inserted and fixed easily into the frame 23 during the assembly process.

### Embodiment 10

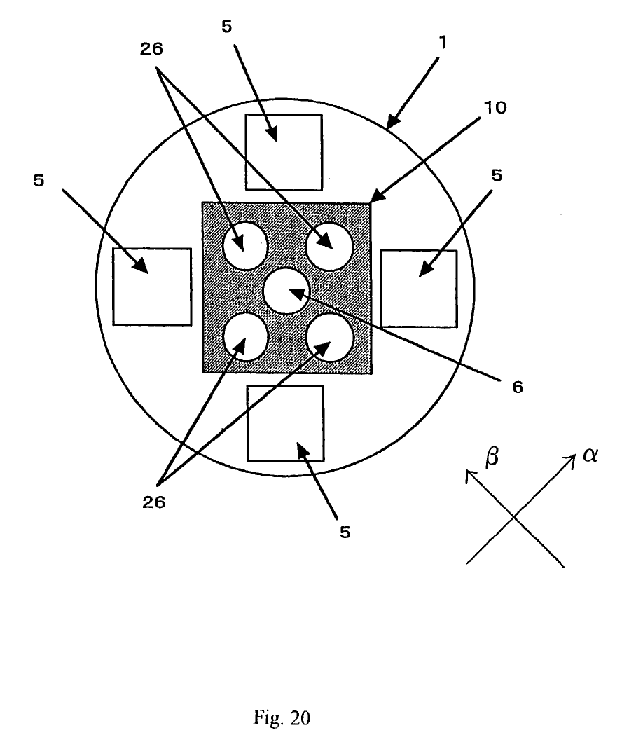

FIG. 20 is a front view as seen from the object side of the capsule endoscope body 1 of Embodiment 10. FIG. 21 is a sectional view of the imaging unit in the “α” direction indicated in FIG. 20. This embodiment uses plural objective optical systems 26, a central objective lens 6, and a single solid-state imaging element 10. An objective optical lens 6 for observation in a direct viewing direction and objective optical systems 26 for observation in a perspective viewing direction are provided. Each objective optical system has a spatial frequency characteristic converter 19.

Generally, it is difficult to control the observation direction of a capsule endoscope body within a patient. Therefore, extending the viewing range of the imaging system is necessary to provide maximum observation and diagnosis within the body. The present embodiment uses plural objective optical systems that have different viewing directions. This allows an extended range of observation within the body. Each objective optical system is provided with a spatial frequency characteristic converter 19. Therefore, the focusing operation is eliminated even though plural objective optical systems are used. As a result, the capsule endoscope body of this embodiment allows an extended range of observation and is easy to assemble.

The image processing unit processes images which are observed from several different directions and are formed on the solid-state imaging element 10 by the plural objective optical system so as to create a single, wide angle image with little distortion. Parallax that occurs when one and the same object is observed by respective objective optical systems can be used to create a three-dimensional image. A capsule endoscope body that combines an imaging unit that includes plural objective optical systems which have different viewing directions with an image processing unit as discussed above, can achieve images that are optimized for the particular observation and diagnosis conditions within the capsule endoscope body. This embodiment uses five objective optical systems, but other numbers of objective optical systems may be used as well.

### Embodiment 11

FIG. 35 shows the eleventh embodiment of the present invention. Whereas Embodiments 1-10 relate to different capsule endoscope body designs, this embodiment relates to a modification of the overall configuration of a capsule endoscope system as shown in FIG. 5. The capsule endoscope body of Embodiment 11 is identical to that discussed in Embodiment 4. The difference in this embodiment relates to the receiver 16 shown in FIG. 5 being simplified by not including the signal processing circuitry 17. Instead, as shown in FIG. 35, the signal processing to restore the spatial frequency content is performed using a personal computer 27.

A TV monitor 28 that is connected via a cable (not illustrated) to the personal computer 27 is used for displaying images of an object obtained using the capsule endoscope to capture the image. Use of the personal computer enables the electronic circuit of the receiver 16 to be simplified from that illustrated in FIG. 5 for Embodiment 4, enabling the power requirements of the receiver to be further reduced, that is to say, for the size of the battery in the receiver 16 to be reduced. Therefore, the receiver 16 is also miniaturized and this serves to reduce the burden of the patient wearing the receiver while he or she is under examination. In this case, version updates of software used for restoring the spatial frequency content can be easily accomplished by using a CD-ROM drive that is available on the personal computer, or the like. In addition, version updates can be accomplished easily in other ways, such as via the Internet or by using the software that is provided with a newly purchased desktop computer or a laptop computer that can be easily carried by a doctor. Providing for convenient version updates enables the software for a capsule endoscope observation system to be tailored to different applications, enabling it to better satisfy the various demands of different doctors in different specialties.

In this figure, the receiver 16 and the personal computer 27, which functions as an image data processing apparatus that restores the spatial frequency content, are electrically connected via a cable 29 that carries the image data signals from the receiver 16. However, a wireless transmission arrangement can be used for the same purpose. Alternatively, any of various known data storing mediums can be used to physically transfer the image data from the receiver 16 to the image data processing apparatus.

### EXAMPLES OF OBJECTIVE LENS SYSTEMS

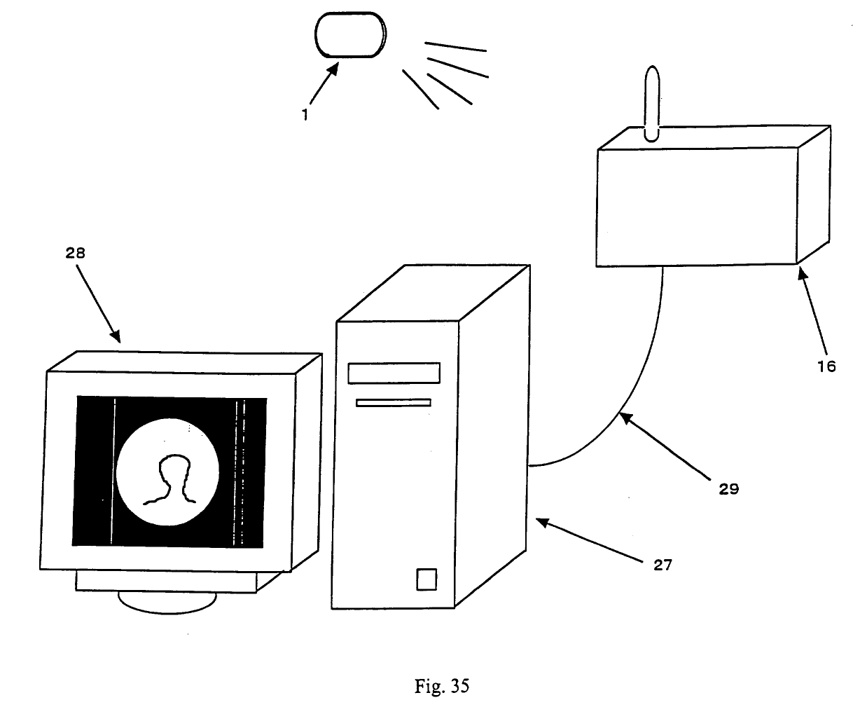

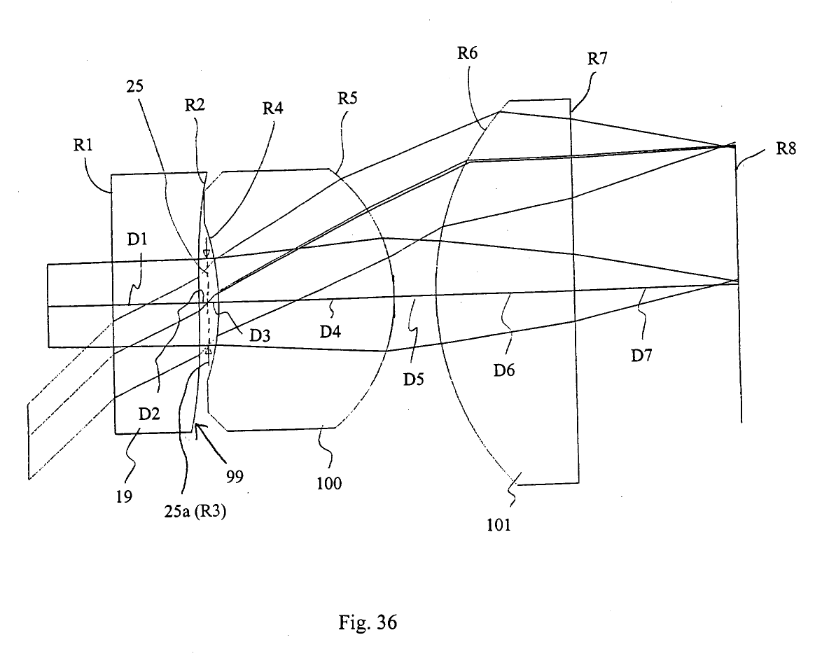

FIG. 36 shows a cross-sectional view of a first example of an objective lens system that may be used in any of the capsule endoscope bodies as discussed above relative to Embodiments 1-10 of the present invention.

The objective lens system of this example is formed of, in order form the object side, a spatial frequency characteristic converter 19 that serves as a pupil modulating element, an aperture stop 25a having an aperture 25, a positive meniscus lens element 100 and a plano-convex lens element 101. The spatial frequency characteristic converter 19 has an aspherical surface 99 on its image-side surface, the shape of which is defined by the following Equation (A):

Z=0.221(x³+y³) Equation (A)

where

Z is the optical axis, and

x and y are two orthogonal axes in a plane perpendicular to the Z axis, with the origin of the coordinates being on the x-y plane.

The aspherical surface is positioned substantially at the pupil plane and operates as a pupil modulating element.

Table 1 below lists the surface number #, in order from the object side, the radius of curvature R (in mm) of each surface near the optical axis, the on-axis spacing D between surfaces, as well as the index of refraction N_{d} and the Abbe number υ_{d} (both measured with respect to the d-line) of the objective lens system shown in FIG. 36. In the Table, the * to the right of surface #2 (i.e. the pupil) indicates that this surface is aspherical, having a shape defined by Equation (A) above.

TABLE 1

| # | R | D | N_{d} | υ_{d} |
|---|---|---|---|---|
| 1 | ∞ | 0.4039 | 1.58900 | 61.3 |
| 2* | ∞ (pupil) | 0.0404 | | |
| 3 | ∞ (stop) | 0.0548 | | |
| 4 | −1.1342 | 0.8346 | 1.58900 | 61.3 |
| 5 | −0.7685 | 0.2019 | | |
| 6 | 1.2789 | 0.6596 | 1.58900 | 61.3 |
| 7 | ∞ | 0.7673 | | |
| 8 | ∞ (image surface) | | | |

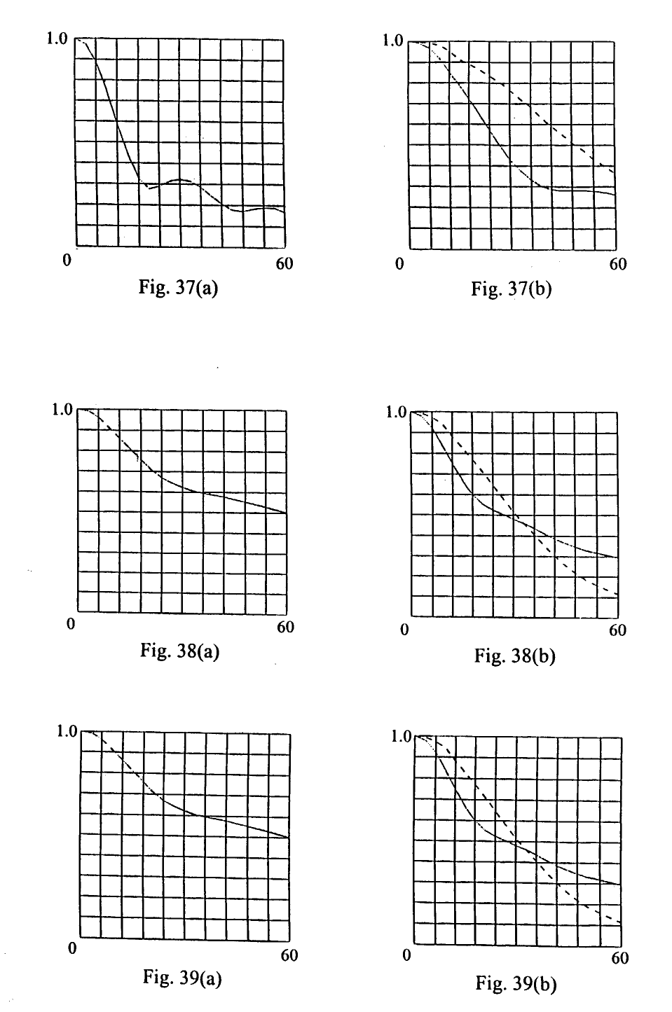

FIGS. 37(a) and 37(b) are graphs of the OTF of the objective lens system set forth in Table 1 that may be used in the present invention, measured on-axis and at the maximum image height, respectively, when the object distance is 5 mm, FIGS. 38(a) and 38(b) are graphs of the OTF, on-axis and at the maximum image height, respectively, when the object distance is 13.5 mm, and FIGS. 39(a) and 39(b) are graphs of the OTF, on-axis and at the maximum image height, respectively, when the object distance is 100 mm. In each of these figures, the Y-axis is the normalized percent transmission and the X-axis is the spatial frequency at the image plane, in line pairs per mm.

In the case where an image pickup device has a pixel pitch of 8 μm, the Nyquist frequency of such a device is 63 line pairs/mm. By extrapolating the illustrated curves out to 63 line pairs per mm, it is apparent from FIGS. 37(a)-39(b), that this objective lens system still has a significant spatial frequency response at the Nyquist frequency for such an image pickup device. Therefore, the OTF can be restored by using a signal processing means for restoring the spatial frequency of the image data modified by the pupil modulating element. In this example, the maximum image height is 0.6 mm.

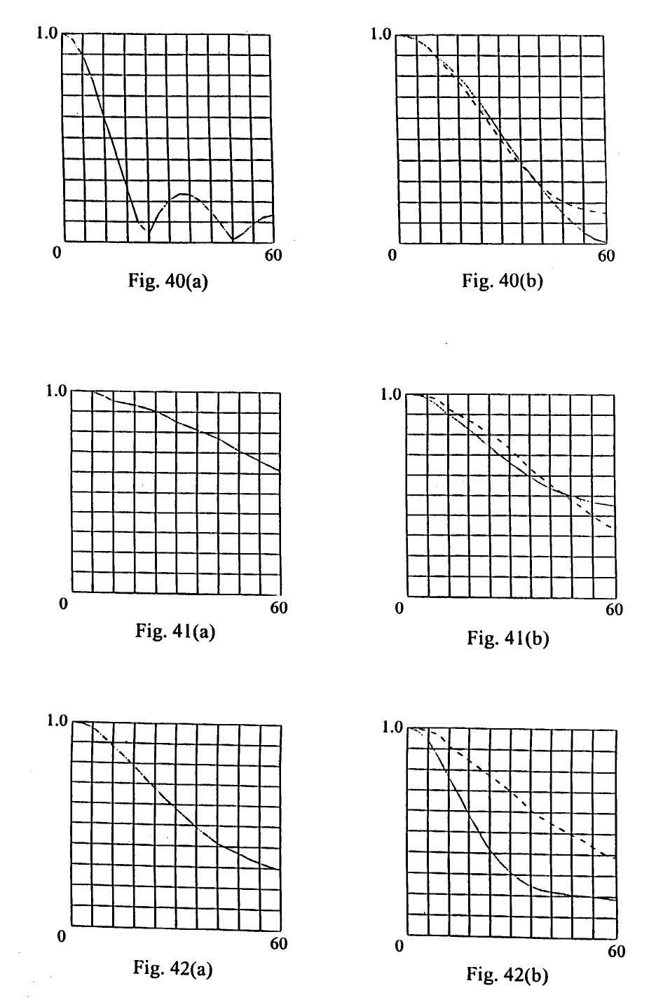

FIGS. 40(a)-42(b) are graphs of the OTF for an objective lens system that is identical to that given in Table 1 above except that surface #2 is planar rather than aspherical. FIGS. 40(a) and 40(b) are graphs of the OTF on-axis and at the maximum image height, respectively, when the object distance is 5 mm, FIGS. 41(a) and 41(b) are graphs of the OTF on-axis and at the maximum image height, respectively, when the object distance is 13.5 mm, and FIGS. 42(a) and 42(b) are graphs of the OTF on-axis and at the maximum image height, respectively, when the object distance is 100 mm. Again, in each of these figures, the Y-axis is the normalized percent transmission and the X-axis is the spatial frequency at the image plane, in line pairs per mm.

As shown in FIG. 40(b), the OTF becomes almost zero at spatial frequencies of 60 line pairs per mm and higher, and therefore, restoring the OTF profile will not be possible by signal processing the converted image signals. In addition, phase inversion occurs and spurious resolution is produced. Further, as the variation of OTF of this objective lens system that depends on object distance is larger than that of the objective lens system having the spatial frequency converter, a processing means that is suitable for one object distance would not be applicable to another object distance.

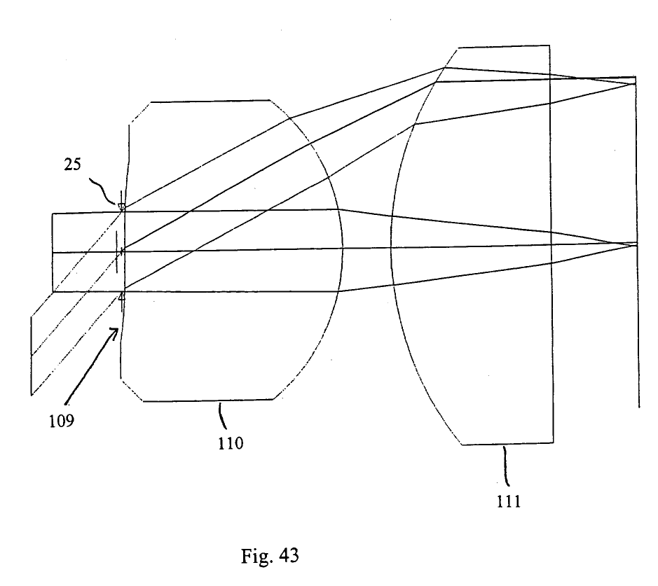

FIG. 43 shows a cross-sectional view of a second example of an objective lens system that may be used in any of the capsule endoscope bodies as discussed above relative to Embodiments 1-10 of the present invention. This objective lens system is formed of, in order from the object side, an aperture 25 that serves as a brightness stop, a positive lens element 110 and a plano-convex lens element 11. The positive lens element 110 has an aspherical surface 109 on its object-side surface, the shape of which is expressed by the following Equation (B):

Z=0.291(X³+y³) Equation (B).

This aspherical surface is positioned at the pupil plane and operates as a spatial frequency characteristic converter.

Table 2 below lists the surface number #, in order from the object side, the radius of curvature R (in mm) of each surface near the optical axis, the on-axis spacing D between surfaces, as well as the index of refraction N_{d} and the Abbe number υ_{d} (both measured with respect to the d-line) of the objective lens system shown in FIG. 43. In the Table, the * to the right of surface #2 (i.e., the pupil) indicates that this surface is aspherical, having a shape defined by Equation (B) above.

TABLE 2

| # | R | D | N_{d} | υ_{d} |
|---|---|---|---|---|
| 1 | ∞ (stop) | 0.0155 | | |
| 2* | ∞ (pupil) | 1.0371 | 1.58900 | 61.3 |
| 3 | −0.8901 | 0.2326 | | |
| 4 | 1.4733 | 0.7599 | 1.58900 | 61.3 |
| 5 | ∞ | 0.4032 | | |
| 6 | ∞ (image surface) | | | |

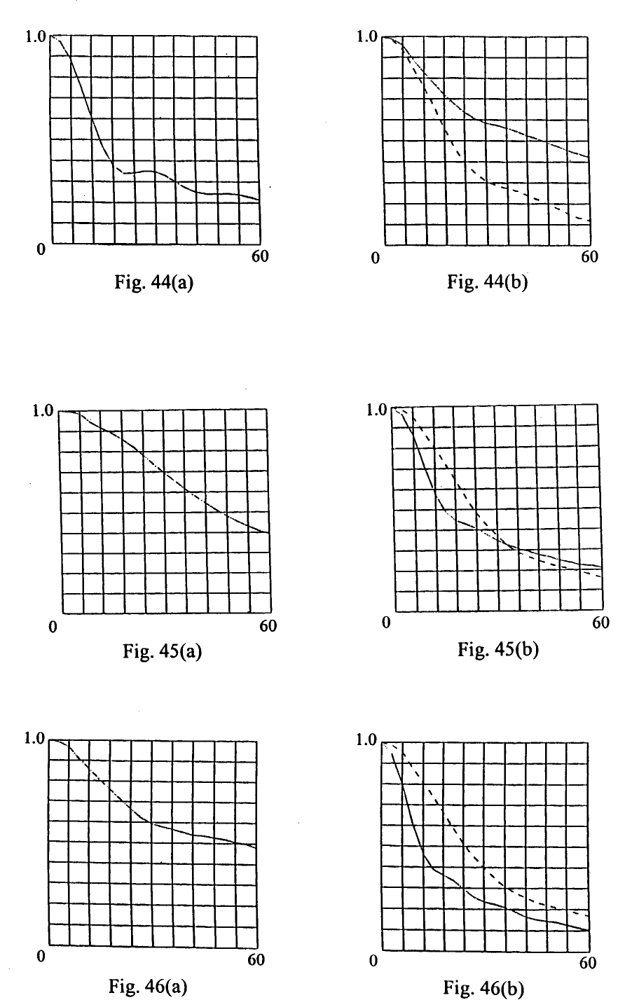

FIGS. 44(a) and 44(b) are graphs of the OTF, measured on-axis and at the maximum image height, respectively, of the objective lens set forth in Table 2 when the object distance is 5 mm, FIGS. 45(a) and 45(b) are graphs of the OTF, measured on-axis and at the maximum image height, respectively, when the object distance is 15.5 mm, and FIGS. 46(a) and 46(b) are graphs of the OTF, measured on-axis and at the maximum image height, respectively, when the object distance is 100 mm. In each of these figures, the Y-axis is the normalized percent transmission and the X-axis is the spatial frequency at the image plane, in line pairs per mm.

In the case where an image pickup device has a pixel pitch of 8 μm, the Nyquist frequency of the device is 63 line pairs/mm. By extrapolating the illustrated curves out to 63 line pairs per mm, it is apparent from FIGS. 44(a)-46(b), that this objective lens system still has a significant spatial frequency response at the Nyquist frequency for such an image pickup device. Therefore, the OTF profile can be restored by using a signal processing means for restoring the spatial frequency of the image data modified by the pupil modulating element at surface #2. In this example, the maximum image height is 0.775 mm.

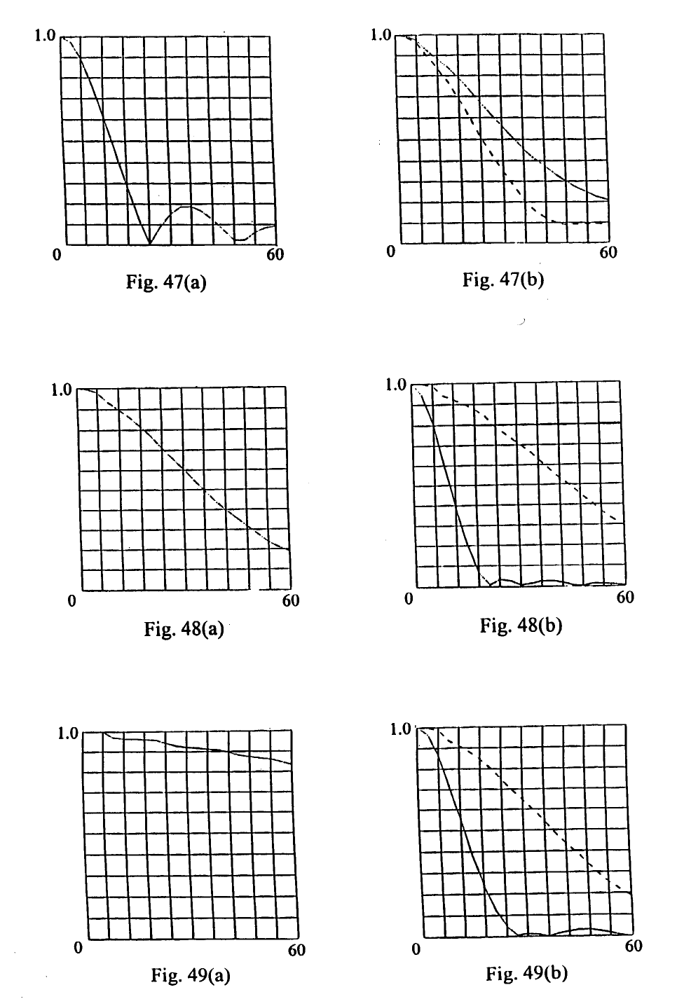

FIGS. 47(a)-49(b) are for an objective lens system that is identical to that given in Table 2 above except that surface #2 is planar rather than aspherical. FIGS. 47(a) and 47(b) are graphs of the OTF, measured on-axis and at the maximum image height, respectively, when the object distance is 5 mm, FIGS. 48(a) and 48(b) are graphs of the OTF, measured on-axis and at the maximum image height, respectively, when the object distance is 13.5 mm, and FIGS. 49(a) and 49(b) are graphs of the OTF, measured on-axis and at the maximum image height, respectively, when the object distance is 100 mm. In each of these figures, the Y-axis is the normalized percent transmission and the X-axis is the spatial frequency at the image plane, in line pairs per mm.

As shown in FIGS. 48(b) and 49(b), the OTF is almost zero for off-axis image points (i.e., significant image heights) at spatial frequencies of well less than 60 line pairs per mm and higher, and therefore, restoring the OTF profile will not be possible by signal processing the converted image signals. In addition, phase inversion occurs and spurious resolution is produced. Further, as the variation of OTF of this objective lens system that depends on object distance is larger than that of the objective lens system having the spatial frequency converter, a processing means that is suitable for one object distance would not be applicable to another object distance.

The advantages of the invention will now be summarized. The capsule endoscope of the present invention can be continuously powered from an external source, solving the problem of a shortage of power within capsule endoscope bodies. This allows an extended observation time for diagnosis within the body. Furthermore, all of the objective optical systems of Embodiments 1 to 11 can use plastic lenses. As described above, the imaging system of the present invention can solve the problem that the prior art objective optical systems of capsule endoscopes suffer from, namely, deteriorated imaging performance and failure to focus because of the temperature and moisture variations in the body. Using plastic lenses with a spatial frequency characteristic converter leads to a reduction in the weight of the optical systems, achieving a light-weight capsule endoscope. This facilitates location control by magnetic induction of the capsule endoscope body within a patient. Plastic lenses also contribute significantly to reducing production costs, and the reduced production costs enable the capsule endoscope body to be discarded after a single use.

The invention being thus described, it will be obvious that the same may be varied in many ways. For example, each of the power units 14 of Embodiments 1 to 11 of the present invention can at least in part be powered by an external power source, with the energy being carried to the capsule endoscope body via electromagnetic waves, such as microwaves. An antenna 15 for receiving microwaves may be provided, as well as a power unit 14 which transforms the microwaves into electrical energy which is then stored in a known device, such as a capacitor or rechargeable battery. The capsule can be further downsized by sharing the antenna 15 with other functions such as image transmission. In this way electric energy can be continuously supplied to the capsule and the energy storage device can be made smaller, which enables the capsule body to be made smaller. Further, the objective optical system can include glass lenses so long as they do not include harmful substances such as arsenic and lead, as these substances would be unsafe for use within a human body and may pose disposal problems. Such variations are not to be regarded as a departure from the spirit and scope of the invention. Rather, the scope of the invention shall be defined as set forth in the following claims and their legal equivalents. All such modifications as would be obvious to one skilled in the art are intended to be included within the scope of the following claims.

What is claimed is:

1. A capsule endoscope body comprising the following components that are sealed within the capsule endoscope body:
an illumination system for illuminating an interior part of a living body;
an imaging system for imaging the interior part that is illuminated by the illumination system, said imaging system including
an objective optical system,
a spatial frequency characteristic converter which causes the optical transfer function of the imaging system to remain essentially constant within some range of in-focus position, and
an image sensor which scans the images so as to convert the images into electrical output signals; and
a transmitter for transmitting the signals output by the image sensor.

2. The capsule endoscope body according to claim 1, wherein the objective optical system consists of a single aspherical lens of positive refractive power.

3. The capsule endoscope body according to claim 1, wherein the objective optical system is formed of only two lens groups, each of positive refractive power.

4. The capsule endoscope body according to claim 1, wherein the objective optical system is formed of, in order from the object side, a first lens group having an overall negative refractive power, and a second lens group having an overall positive refractive power.

5. The capsule endoscope body according to claim 1, wherein the spatial frequency characteristic converter has an aperture which is shaped the same as the light receiving part of the image sensor.

6. The capsule endoscope body according to claim 1, wherein the image sensor is an MOS-type image sensor.

7. The capsule endoscope body according to claim 1, wherein power for operating the capsule endoscope body is provided by at least one battery located within the sealed capsule endoscope body.

8. The capsule endoscope body according to claim 1, wherein power for operating the capsule endoscope body is provided at least in part by the capsule endoscope body receiving electromagnetic energy from an energy source which is located outside the sealed capsule endoscope body.

9. The capsule endoscope body according to claim 1, wherein the objective optical system is formed of plastic lenses.

10. A capsule endoscope body according to claim 1, and further including within said capsule endoscope body a signal processing circuit for adjusting for manufacturing variations of the imaging system.

11. A capsule endoscope receiver for receiving image data transmitted from a capsule endoscope body, said image data having been modified by a spatial frequency characteristic converter which causes the optical transfer function of an imaging system to remain essentially constant within some range of in-focus position, said capsule endoscope receiver including a signal processor for restoring the spatial frequency content of the modified image data.

12. In combination: a capsule endoscope body according to claim 1 and a capsule endoscope receiver for receiving image data transmitted from the capsule endoscope body, said image data having been modified by a spatial frequency characteristic converter which causes the optical transfer function of the imaging system to remain essentially constant within some range of in-focus position, said capsule endoscope receiver including a signal processor for restoring the spatial frequency content of the modified image data.

13. In combination: a capsule endoscope body according to claim 10 and a capsule endoscope receiver for receiving image data transmitted from the capsule endoscope body.

14. The capsule endoscope body according to claim 1, wherein the sealed capsule has a transparent, substantially oval-shaped, tip portion cover that covers the front of the objective optical system and the illumination system.

15. The capsule endoscope body according to claim 1, wherein:
the sealed capsule has a transparent tip portion cover that covers the front of the objective optical system and the illumination system, with the shape of the tip portion cover at a part that covers the front of the objective optical system being different from the shape of the tip portion cover at a part that covers the front of the illumination system.

16. The capsule endoscope body according to claim 1, wherein the spatial frequency characteristic converter is positioned substantially at a pupil of the objective optical system.

17. The capsule endoscope body according to claim 1, wherein a frame for housing the objective optical system has an opening on the object side of the objective optical system which serves as a brightness stop, and the spatial frequency characteristic converter is positioned substantially at the brightness stop.

18. The capsule endoscope body according to claim 2, wherein a frame for housing the objective optical system has an opening on the object side of the objective optical system which serves as a brightness stop, and the spatial frequency characteristic converter is positioned substantially at the brightness stop.

19. The capsule endoscope body according to claim 3, wherein a frame for housing the objective optical system has an opening on the object side of the objective optical system which serves as a brightness stop, and the spatial frequency characteristic converter is positioned substantially at the brightness stop.

20. The capsule endoscope body according to claim 1, wherein said spatial frequency characteristic converter has a circular aperture.

21. The capsule endoscope body according to claim 1, wherein the imaging system further includes an additional objective optical system.

22. The capsule endoscope body according to claim 1, wherein said image sensor is a CCD.

23. In combination: a capsule endoscope body according to claim 1, a capsule endoscope receiver for receiving image data transmitted from the capsule endoscope body, and an image data processing apparatus for processing the image data and displaying the image on a monitor, the image having been modified by a spatial frequency converter which causes the optical transfer function of the imaging system to remain essentially constant within some range of in-focus position, the image data processing apparatus including a signal processor for restoring the spatial frequency content of the modified image data.
